(19) United States
(12) Patent Application Publication
(10) Pub. No.: US 2022/0202879 A1
(43) Pub. Date: Jun. 30, 2022

(54) COMPOSITIONS AND METHODS OF TREATING ASTHMA

(71) Applicants: The Jackson Laboratory, Bar Harbor, ME (US); Washington University, St. Louis, MO (US)

(72) Inventors: Yanjiao Zhou, Farmington, CT (US); Avraham Beigelman, Raanana (IL); Leonard Bacharier, Nashville, TN (US); George Weinstock, Avon, CT (US)

(73) Assignees: The Jackson Laboratory, Bar Harbor, ME (US); Washinton University, St. Louis, MO (US)

(21) Appl. No.: 17/551,057

(22) Filed: Dec. 14, 2021

Related U.S. Application Data
(60) Provisional application No. 63/125,714, filed on Dec. 15, 2020.

(51) Int. Cl. A61K 35/741; A61P 11/06

(52) U.S. Cl. CPC A61K 35/741; A61P 11/06

(57) ABSTRACT

The present disclosure relates to methods and compositions utilizing upper-airway microbiota for diagnosing individuals at risk for asthma exacerbations. Further provided herein are probiotic compositions and methods for treating asthma by administering microorganisms that are associated with decreased risk of asthma exacerbations.

### RELATED APPLICATION

This application claims the benefit under 35 U.S.C. § 119(e) of U.S. provisional application No. 63/125,714 filed Dec. 15, 2020, which is incorporated by reference herein in its entirety.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Dec. 14, 2021, is named J022770103US01-SEQ-NTJ, and is 7,329 bytes in size.

### TECHNICAL FIELD

Provided in certain instances here are methods and compositions utilizing upper-airway microbiota for diagnosing individuals at risk for asthma exacerbations. Further provided herein are compositions and methods for treating asthma by administering microorganisms that are associated with decreased risk of asthma exacerbations.

### BACKGROUND

Asthma exacerbations have high impact on children, their families, the health care system, and may lead to subsequent decline in lung function. Early signs of loss of asthma control, often referred to as the Yellow Zone (YZ), is a period during which the patient is at risk of symptom progression to severe exacerbation. Among preschool children, the airway microbiome is associated with respiratory illness severity, future wheezing, and childhood asthma. However, it is unknown if the airway microbiome is related to asthma control and risk of exacerbations among school-age children with mild asthma. Therefore, there is a need for more efficient and safer strategies for identifying individuals at risk of asthma exacerbations and/or loss of asthma control, and for effectively treating such individuals, especially children.

### SUMMARY

Described herein are compositions and methods for identifying individuals at risk of loss of asthma control and/or asthma exacerbations. The compositions and methods utilize the nasal microbiome as a biomarker of asthma exacerbations and/or loss of asthma control. Described herein are compositions and methods for treating or controlling asthma exacerbations and/or loss of asthma control by administering microorganisms that are associated with a decreased risk of asthma exacerbations.

The present disclosure is based on the discovery that the airway microbiome plays a role in asthma pathophysiology. The present disclosure demonstrates that airway microbiota colonization patterns in asthma patients are associated with a risk of loss of asthma control and/or severe exacerbations. For example, airway microbiota dominated by Corynebacterium+Dolosigranulum genera are associated with favorable clinical outcomes compared to microbiota dominated by more pathogenic Staphylococcus, Streptococcus, and Moraxella bacteria. As demonstrated herein, nasal blow samples were collected from children with asthma at two time points: randomization (RD) at a time when asthma is under control, and later at a time of early loss of asthma control (designed herein “Yellow Zone” (YZ)). Participants whose microbiota were dominated by the commensal Corynebacterium+Dolosigranulum cluster at RD experienced the lowest rate of YZ, and/or had the longest time to develop YZ. Furthermore, the airway microbiota changed from randomization to YZ. A switch from the Corynebacterium+Dolosigranulum cluster at randomization to the Moraxella cluster at YZ posed the highest risk of severe asthma exacerbation. Finally, Corynebacterium's relative abundance at YZ was inversely associated with severe exacerbations. The results demonstrate the utility of nasal microbiome as a biomarker to predict asthma exacerbations, and/or treat individuals at risk for asthma exacerbations, e.g., in individuals showing early signs of loss of asthma control.

Thus, in some aspects, provided herein is a method comprising: (a) acquiring a biological sample from an upper airway of an individual with early signs of loss of asthma control; (b) identifying a plurality of microorganisms in the biological sample; and (c) determining an amount, concentration, or proportion of at least one microorganism in the biological sample.

In some embodiments, the early signs of loss of asthma control comprise increased use of asthma intervention therapy or increased asthma symptoms.

In some embodiments, the individual with early signs of loss of asthma control used at least one dose of albuterol and remained awake for one night due to asthma symptoms, used at least two doses of albuterol in 6 hours, used at least two inhalations of albuterol in 6 hours, used at least three doses of albuterol in 24 hours, or used at least 6 inhalations of albuterol in 24 hours.

In some embodiments, the microorganism is selected from the group consisting of microorganisms of genus Moraxella, microorganisms of genus Staphylococcus, microorganisms of genus Streptococcus, microorganisms of genus Corynebacterium, and microorganisms of genus Dolosigranulum.

In some aspects, provided herein is a method comprising: (a) acquiring a biological sample from an upper airway of an individual with asthma, (b) analyzing a plurality of polynucleotides extracted from the biological sample, and (c) determining an amount, concentration, or proportion of a microorganism in the biological sample, wherein the microorganism is selected from the group consisting of microorganisms of genus Staphylococcus, microorganisms of genus Streptococcus microorganisms of genus Corynebacterium, and microorganisms of genus Dolosigranulum.

In some aspects, provided herein is a method comprising (a) acquiring a biological sample from an upper airway of an individual with asthma, (b) analyzing a plurality of polynucleotides extracted from the biological sample, and (c) determining an amount, concentration, or proportion of a microorganism in the biological sample, wherein the microorganism is selected from the group consisting of microorganisms of Moraxella species M. catarrhalis, M. nonliquefaciens, or M. lincolnii, microorganisms of genus Staphylococcus, microorganisms of genus Streptococcus, microorganisms of genus Corynebacterium, and microorganisms of genus Dolosigranulum.

In some embodiments, the microorganisms of Moraxella species comprise 16S RNA sequences with 99% identity to any one of SEQ ID NOs. 1-3.

In some embodiments, the microorganisms of genus Staphylococcus, comprise microorganisms of Staphylococcus species S. aureus or S. epidermidis.

In some embodiments, the microorganisms of Staphylococcus species comprise 16S RNA sequences with 99% identity to any one of SEQ ID NOs. 4-5.

In some embodiments, the microorganisms of genus Streptococcus, comprise microorganisms of Streptococcus species S. pneumoniae.

In some embodiments, the microorganisms of Streptococcus species comprise 16S RNA sequences with 99% identity to SEQ ID NO. 6.

In some embodiments, the microorganisms of genus Corynebacterium comprise microorganisms of Corynebacterium species C. pseudodiphtheriticum or C. accolens.

In some embodiments, microorganism of genus Corynebacterium comprise 16S RNA sequences with 99% identity to any one of SEQ ID NOs. 7-8.

In some embodiments, the microorganisms of genus Dolosigranulum comprise microorganisms of Dolosigranulum species D. pigrum.

In some embodiments, the microorganism of genus Dolosigranulum comprise 16S RNA sequences with 99% identity to SEQ ID NO. 9.

In some embodiments, the individual does not exhibit asthma symptoms.

In some embodiments, step (c) comprises determining an amount, concentration, or proportion of two or more microorganisms.

In some embodiments, step (c) comprises determining an amount, concentration, or proportion of three or more microorganisms.

In some embodiments, step (b) comprises analyzing a plurality of polynucleotides extracted from the biological sample.

In some embodiments, step (b) comprises detecting a 16S rRNA sequence from the polynucleotides.

In some embodiments, step (b) comprises hybridizing a polynucleotide to an oligonucleotide array.

In some embodiments, step (b) comprises hybridizing a polynucleotide to an oligonucleotide bait. In some embodiments, the oligonucleotide bait comprises a 16S rRNA sequence.

In some embodiments, step (b) comprises amplification of a segment of a polynucleotide.

In some embodiments, the segment of the polynucleotide comprises a 16S rRNA sequence.

In some embodiments, step (b) comprises sequencing a polynucleotide or a segment of a polynucleotide.

In some embodiments, step (c) comprises determining that a sequence of a polynucleotide has at least about 99% identity to a reference sequence of the microorganism.

In some embodiments, step (c) comprises comparing a polynucleotide sequences of bacteria by analyzing a 16S rRNA sequence with a DESeq2 algorithm.

In some embodiments, step (b) comprises analyzing a plurality of polypeptides extracted from the biological sample.

In some embodiments, step (b) comprises performing matrix desorption/ionization time of flight mass spectrometry (MALDI-TOF).

In some embodiments, step (b) comprises performing Whole Genome Sequencing (WGS).

In some aspects, provided herein is a method comprising: (a) acquiring a first biological sample from an individual with asthma at a time when the individual does not manifest symptoms of asthma; (b) acquiring a second biological sample from the individual with asthma at a time when the individual manifests early signs of loss of asthma control; and (c) comparing a microbiome of the first biological sample to a microbiome of the second biological sample.

In some embodiments, the individual with early signs of loss of asthma control: used at least one dose of albuterol and remained awake for one night due to asthma symptoms, used at least two doses of albuterol in 6 hours, used at least two inhalations of albuterol in 6 hours, used at least three doses of albuterol in 24 hours, or used at least 6 inhalations of albuterol in 24 hours.

In some embodiments, the first biological sample and the second biological sample are collected from an upper airway of the individual.

In some embodiments, step (c) comprises analyzing a plurality of polynucleotides extracted from the first biological sample and analyzing a plurality of polynucleotides extracted from the second biological sample.

In some embodiments, step (c) comprises analyzing a plurality of polypeptides extracted from the first biological sample and analyzing a plurality of polypeptides extracted from the second biological sample.

In some embodiments, an increase in microorganisms of genus Moraxella in the second biological sample and a decrease in microorganisms of genus Corynebacterium, and genus Dolosigranulum in the second biological sample is indicative of an increased risk of asthma exacerbations.

In some embodiments, the biological sample is a nasal blow sample.

In some embodiments, the method further comprises determining a presence or absence of a respiratory virus in the biological sample. In some embodiments, the respiratory virus is an enterovirus, a rhinovirus, or a rhinovirus C. In some embodiments, the presence or absence of a respiratory virus is determined by a multiplex polymerase chain reaction.

In some embodiments, the individual is a child of between 2 and 17 years old. In some embodiments, the child is age 5-11 years old. In some embodiments, the child is age 5-7 years old.

In some embodiments, the method further comprises determining if the individual lives in a household with a pet, wherein presence of a pet is indicative of an increased risk of asthma exacerbations.

In some embodiments, the method further comprises determining a respiratory rate of the individual, a respiratory flow rate of the individual, a pulse rate of the individual, or a partial pressure of oxygen in arterial blood of the individual.

In some embodiments, step (c) comprises determining that an amount, concentration, or proportion of the microorganism in the biological sample is altered relative to a baseline an amount, concentration, or proportion of the microorganism in a biological sample acquired from the individual before the onset of early signs of loss of asthma control.

In some embodiments, step (c) comprises determining that an amount, concentration, or proportion of the microorganism in the biological sample is altered relative to an amount, concentration, or proportion of the microorganism a reference biological sample isolated from an individual who has not been diagnosed with asthma.

In some embodiments, an increased amount, concentration, or proportion of a microorganism of Moraxella species selected from M. catarrhalis, M. nonliquefaciens and M. lincolnii, genus Staphylococcus or genus Streptococcus or a decreased amount, concentration, or proportion of a microorganism of genus Corynebacterium or genus Dolosigranulum is indicative of (i) an increased risk of asthma exacerbations; (ii) a decreased time to loss of asthma control or asthma exacerbations; or (iii) an increased risk of loss of asthma control in the subject.

In some aspects, provided herein is a method of treating an individual having early signs of loss of asthma control or asthma exacerbation comprising: (1) detecting an altered amount, concentration, or proportion of a microorganism in a biological sample from an upper airway of the individual; and (2) administering to the individual an agent that is effective to prevent or attenuate asthma exacerbations.

In some embodiments, the early signs of loss of asthma control comprise increased use of asthma intervention therapy or increased asthma symptoms.

In some embodiments, the individual with early signs of loss of asthma control or asthma exacerbations: used at least one dose of albuterol and remained awake for one night due to asthma symptoms, used at least two doses of albuterol in 6 hours, used at least two inhalations of albuterol in 6 hours, used at least three doses of albuterol in 24 hours, or used at least 6 inhalations of albuterol in 24 hours.

In some embodiments, the microorganism is selected from the group consisting of microorganisms of genus Moraxella, microorganisms of genus Staphylococcus, microorganisms of genus Streptococcus microorganisms of genus Corynebacterium, and microorganisms of genus Dolosigranulum.

In some embodiments, the detecting step comprises (a) acquiring a biological sample from an upper airway of an individual with early signs of loss of asthma control or asthma exacerbation, (b) identifying a plurality of microorganisms in the biological sample, and (c) determining an amount, concentration, or proportion of a microorganism in the biological sample.

In some aspects, provided herein is a method of treating an individual with asthma comprising: (1) detecting an increased amount, concentration, or proportion of a microorganism of genus Moraxella, genus Staphylococcus or genus Streptococcus compared to an amount, concentration, or proportion of a microorganism of genus Corynebacterium or genus Dolosigranulum in a biological sample from an upper airway of the individual; and (2) administering to the individual an agent that is effective to prevent or attenuate asthma exacerbations.

In some embodiments, the detecting step comprises (a) acquiring a biological sample from an upper airway of an individual with asthma, (b) identifying a plurality of microorganisms in the biological sample, and (c) determining an amount, concentration, or proportion of a microorganism in the biological sample, wherein the microorganism is selected from the group consisting of microorganisms of genus Moraxella, genus Staphylococcus, microorganisms of genus Streptococcus microorganisms of genus Corynebacterium, and microorganisms of genus Dolosigranulum.

In some aspects, provided herein is a method of treating an individual with asthma comprising: (1) detecting an increased amount, concentration, or proportion of a microorganism of Moraxella M. catarrhalis, M. nonliquefaciens and M. lincolnii, genus Staphylococcus or genus Streptococcus or a decreased amount, concentration, or proportion of a microorganism of genus Corynebacterium or genus Dolosigranulum in a biological sample from an upper airway of the individual; and (2) administering to the individual an agent that is effective to prevent or attenuate asthma exacerbations.

In some embodiments, the detecting step comprises (a) acquiring a biological sample from an upper airway of an individual with asthma, (b) identifying a plurality of microorganisms in the biological sample, and (c) determining an amount, concentration, or proportion of a microorganism in the biological sample, wherein the microorganism is selected from the group consisting of microorganisms of Moraxella species selected from M. catarrhalis, M. nonliquefaciens, and M. lincolnii, microorganisms of genus Staphylococcus, microorganisms of genus Streptococcus microorganisms of genus Corynebacterium, and microorganisms of genus Dolosigranulum.

In some embodiments, step (c) comprises determining an amount, concentration, or proportion of two or more microorganisms.

In some embodiments, step (c) comprises determining an amount, concentration, or proportion of three or more microorganisms.

In some embodiments, step (b) comprises analyzing a plurality of polynucleotides extracted from the biological sample

In some embodiments, step (b) comprises detecting a 16S rRNA sequence.

In some embodiments, step (b) comprises hybridizing a polynucleotide to an oligonucleotide array.

In some embodiments, step (b) comprises hybridizing a polynucleotide to an oligonucleotide bait. In some embodiments, the oligonucleotide bait comprises a 16S rRNA sequence.

In some embodiments, step (b) comprises amplification of a segment of a polynucleotide.

In some embodiments, the segment of the polynucleotide comprises a 16S rRNA sequence.

In some embodiments, step (b) comprises sequencing a polynucleotide or a segment of a polynucleotide.

In some embodiments, step (c) comprises determining that a sequence of a polynucleotide has at least about 99% identity to a reference sequence of the microorganism.

In some embodiments, step (c) comprises comparing a polynucleotide sequences of bacteria by analyzing a 16S rRNA sequence with a DESeq2 algorithm.

In some embodiments, step (b) comprises analyzing a plurality of polypeptides extracted from the biological sample.

In some embodiments, step (b) comprises performing matrix desorption/ionization time of flight mass spectrometry (MALDI-TOF).

In some embodiments, step (b) comprises performing Whole Genome Sequencing (WGS).

In some embodiments, step (c) comprises determining that an amount, concentration, or proportion of the microorganism in the biological sample is altered relative to a baseline an amount, concentration, or proportion of the microorganism in a biological sample acquired from the individual before the onset of early signs of loss of asthma control.

In some embodiments, step (c) comprises determining that an amount, concentration, or proportion of the microorganism in the biological sample is altered relative to an amount, concentration, or proportion of the microorganism a reference biological sample isolated from an individual who has not been diagnosed with asthma.

In some aspects, provided herein is a method of treating an individual with asthma comprising (a) detecting a change in an upper airway microbiome of the individual; and (b) administering a therapeutic composition to the individual; thereby preventing or reducing severity of an asthma exacerbation of the individual.

In some embodiments, the step of detecting a change in an upper airway microbiome of the individual comprises: (i) acquiring a first biological sample from an individual with asthma at a time when the individual does not manifest symptoms of asthma; (ii) acquiring a second biological sample from the individual with asthma at a time when the individual manifests early signs of loss of asthma control; and (iii) comparing a microbiome of the first biological sample to a microbiome of the second biological sample.

In some embodiments, the individual with early signs of loss of asthma control: used at least one dose of albuterol and remained awake for one night due to asthma symptoms, used at least two doses of albuterol in 6 hours, used at least two inhalations of albuterol in 6 hours, used at least three doses of albuterol in 24 hours, or used at least 6 inhalations of albuterol in 24 hours.

In some embodiments, the first biological sample and the second biological sample are collected from an upper airway of the individual.

In some embodiments, step (iii) comprises analyzing a plurality of polynucleotides extracted from the first biological sample and analyzing a plurality of polynucleotides extracted from the second biological sample.

In some embodiments, step (iii) comprises analyzing a plurality of polypeptides extracted from the first biological sample and analyzing a plurality of polypeptides extracted from the second biological sample.

In some embodiments, step (iii) comprises performing Whole Genome Sequencing (WGS).

In some embodiments, an increase in microorganisms of genus Moraxella in the second biological sample and a decrease in microorganisms of genus Corynebacterium, and genus Dolosigranulum in the second biological sample is indicative of an increased risk of asthma exacerbations.

In some embodiments, the agent is administered to an upper airway of the individual.

In some embodiments, the individual has a respiratory tract infection, and wherein the agent is an antibiotic.

In some embodiments, the agent is a prebiotic composition.

In some embodiments, the agent is a probiotic composition.

In some embodiments, the probiotic composition comprises an isolated microorganism of genus Corynebacterium, genus Dolosigranulum, or a combination thereof.

In some embodiments, the isolated microorganism is a purified microorganism.

In some embodiments, the method further comprises a step of administering a second agent that prevents or attenuates asthma exacerbations. In some embodiments, the second agent is a drug, a prebiotic, a bacterial extract or a fungus.

In some embodiments, the method further comprises, prior to administering, determining the individual has a respiratory rate of greater than 60 breaths per minute, a pulse of greater than 120 beats per minute, or a partial pressure of oxygen in arterial blood of less than 60 mM Hg.

In some embodiments, the treatment (a) prevents, delays, or attenuates progression of the individual with asthma from a Green Zone to a Yellow Zone, from a Green Zone to a Red Zone or from a Yellow Zone to a Red Zone, or (b) reduces the time the individual with asthma remains in a Yellow Zone or a Red Zone; or (c) increases the time to at least two episodes of Yellow Zone.

In some aspects, provided herein is a method of modifying a nasal microbiota of an individual comprising administering to the individual a probiotic composition comprising an isolated microorganism at a dose sufficient to cause an increase in an abundance or proportion of the microorganism in the nasal microbiota of the individual, wherein the isolated microorganism is a microorganism of genus Corynebacterium or a microorganism of genus Dolosigranulum.

In some embodiments, the nasal microbiota of the individual comprises: (a) an elevated abundance or proportion relative to a reference sample of one or more microorganisms of genus Moraxella, genus Staphylococcus or genus Streptococcus, or (b) a reduced abundance or proportion relative to the reference sample of one or more microorganisms of genus Corynebacterium or genus Dolosigranulum.

In some aspects, provided herein is a method of treating an individual with a nasal microbiome characterized by (a) an elevated abundance or proportion relative to a reference sample of one or more microorganisms of genus Moraxella, genus Staphylococcus or genus Streptococcus; or (b) a reduced abundance or proportion relative to the reference sample of one or more microorganisms of genus Corynebacterium or genus Dolosigranulum, the method comprising administering to the individual a probiotic composition comprising an isolated microorganism, wherein the isolated microorganism is a microorganism of genus Corynebacterium or genus Dolosigranulum, thereby preventing, delaying or attenuating an asthma exacerbation of the individual.

In some aspects, provided herein is a method of treating an individual with a nasal microbiome characterized by (a) an elevated abundance or proportion relative to a reference sample of one or more microorganisms of genus Moraxella, genus Staphylococcus or genus Streptococcus; or (b) a reduced abundance or proportion relative to the reference sample of one or more microorganisms of genus Corynebacterium or genus Dolosigranulum, the method comprising administering to the individual a probiotic composition comprising an isolated microorganism, wherein the isolated microorganism is a microorganism of genus Corynebacterium or genus Dolosigranulum, thereby preventing or treating an upper respiratory tract infection of the individual.

In some embodiments, the reference sample is a biological sample obtained from an upper airway of the individual before the onset of early signs of loss of asthma control.

In some embodiments, the reference sample is a biological sample obtained from an upper airway of an individual not diagnosed with asthma.

In some aspects, provided herein is a bacterial extract for administration to an upper airway of an individual with asthma, wherein the bacterial extract is: (a) isolated from a microorganism of genus Corynebacterium or genus Dolosigranulum, and (b) capable of inhibiting growth and/or colonization of the upper airway of the individual by a microorganism of genus Staphylococcus, genus Streptococcus, or genus Moraxella.

In some embodiments, the microorganisms of genus Corynebacterium comprise microorganisms of Corynebacterium species C. pseudodiphtheriticum or C. accolens.

In some embodiments, the microorganism of genus Corynebacterium comprise 16S RNA sequences with 99% identity to any one of SEQ ID NOs. 7-8.

In some embodiments, the microorganisms of genus Dolosigranulum comprise microorganisms of Dolosigranulum species D. pigrum.

In some embodiments, wherein the microorganism of genus Dolosigranulum comprise 16S RNA sequences with 99% identity to SEQ ID NO. 9.

In some embodiments, the bacterial extract is secreted by the microorganism of genus Corynebacterium or genus Dolosigranulum.

In some embodiments, the bacterial extract is isolated from an outer surface of the microorganism of genus Corynebacterium or genus Dolosigranulum.

In some aspects, provided herein is a method of treating an individual comprising administering the bacterial extract of any one of claims 100-106 to an upper airway of the individual, thereby reducing an amount or proportion of a microorganism of genus Staphylococcus, genus Streptococcus, or genus Moraxella in an upper respiratory tract of the individual.

In some embodiments, the individual is an individual with asthma, thereby preventing, delaying or attenuating progression of the individual with asthma from a Green zone to a Yellow Zone, from a Green Zone to a Red Zone or from a Yellow Zone to a red Zone, or wherein the treatment reduces the time the individual with asthma remains in a Yellow Zone or a Red Zone.

In some aspects, provided herein is a probiotic formulation for preventing, delaying or attenuating asthma exacerbations comprising a pharmaceutically acceptable unit dose of a probiotic composition comprising one or more isolated microorganisms selected from microorganisms of genus Corynebacterium or genus Dolosigranulum, and a pharmaceutically acceptable carrier.

In some embodiments, the microorganisms of genus Corynebacterium comprise microorganisms of Corynebacterium species C. pseudodiphtheriticum or C. accolens.

In some embodiments, the microorganism of genus Corynebacterium comprise 16S RNA sequences with 99% identity to any one of SEQ ID NOs. 7-8.

In some embodiments, the microorganisms of genus Dolosigranulum comprise microorganisms of Dolosigranulum species D. pigrum.

In some embodiments, the microorganism of genus Dolosigranulum comprise 16S RNA sequences with 99% identity to SEQ ID NO. 9.

In some embodiments, the pharmaceutically acceptable unit dose is formulated for nasal administration.

In some embodiments, the probiotic formulation further comprises a propellant.

In some aspects, provided herein is a probiotic formulation comprising a bacterial extract as described herein, and a pharmaceutically acceptable carrier.

The present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings, wherein:

FIGS. 1A-1C: Top 25 major taxa identified at randomization. Top 25 most abundant taxa in different age groups (FIG. 1A) and by the presence of respiratory virus in the sample (FIG. 1B) are shown by bar plots. The Odd Ratio of developing YZ is displayed with 95% confidence interval (FIG. 1C).

FIG. 2: Bacterial clusters identified in nasal samples at randomization (n=214). Corynebacterium+Dolosigranulum), Staphylococcus, Streptococcus, and Moraxella were identified by hierarchical clustering and complete linkage approach. Bacterial genera with relative abundance >0.1% was used for clustering analysis. Age group, presence of respiratory virus in the sample, and the annualized yearly rate of Yellow Zone (YZ) episodes are annotated along each cluster.

FIGS. 3A-3D: Yearly rate of Yellow Zone (YZ) in respiratory clusters at randomization. (FIG. 3A) Yearly rate of YZ in Corynebacterium+Dolosigranulum cluster compared to the combined Others 3 clusters. The line in the middle of a boxplot represents the median value of Yearly rate of YZ; the line at bottom and top of a box represent 25 and 75 percentile of the data value. Dots above the vertical line represent outliers of the data. (FIG. 3B) Yearly rate of YZ in the four respiratory clusters. In FIGS. 3A and 3B, each dot represents a participant in the cluster. Statistical significance of YZ rate differences between clusters was tested using Wilcoxon rank-sum test. (FIG. 3C) Corynebacterium+Dolosigranulum cluster has significantly lower probability than the combined other three clusters (Other) to develop >=2 episode of YZ (p=0.02 by Cox Proportional-Hazards model). (FIG. 3D) Corynebacterium+Dolosigranulum cluster has significantly lower probability than the Moraxella cluster and the Staphylococcus cluster to develop >=2 episodes of YZ (p=0.05 by Cox Proportional-Hazards model).

FIGS. 4A-4C: Bacterial microbiome comparison between randomization (RD) and Yellow Zone (YZ). (FIG. 4A) Five bacterial clusters were identified using the paired randomization (n=102) and YZ data (n=102). Each bar plot represents the proportion of patients belonging to a given cluster. Green-samples collected at RD, and orange-samples collected at YZ. Sums of proportions for green/orange bars is equal to 100%. The statistical difference of patient distribution across the five clusters was identified using Chi-Square test. (FIG. 4B) Changes in the relative abundance of bacterial microbiome from RD to YZ in each of the study participants. The relative abundance of the top 25 bacteria from RD (top panel) and YZ (bottom panel) in the same subjects are plotted. The samples are ordered by clusters at RD from left to right (1) Corynebacterium+Dolosigranulum (2) Haemophilus (3) Moraxella (4) Staphylococcus (5) Streptococcus clusters. (FIG. 4C) Total bacterial load in RD and YZ samples. Total bacterial load is represented as 16S rRNA million copies per μL, estimated by qPCR. (FIG. 4D) Bacterial richness at RD and YZ. Statistical difference of bacterial load or richness was tested using Wilcoxon rank-sum test.

FIG. 5: Changes in bacterial clusters from randomization to Yellow Zone (YZ). The numbers within each cluster at RD (left) and YZ (right) are the number of patients within each specific cluster switch.

FIG. 6: Bacterial clusters identified from 105 nasal samples at Yellow Zone (YZ). Five clusters, Corynebacterium+Dolosigranulum, Staphylococcus, Streptococcus, Moraxella, and Haemophilus were identified by hierarchical clustering and complete linkage approach. Bacterial genera with relative abundance >0.1% was used for clustering analysis.

FIGS. 7A-7B: The microbiome at YZ and the outcome of severe exacerbations (OCS treatment). (FIG. 7A) The proportion of patients progressed to severe exacerbation (OCS therapy, n=30) across different bacterial clusters at YZ. (FIG. 7B) The relative abundance of Corynebacterium at the time of YZ is significantly lower in patients that progressed to severe exacerbation (P=0.002, Deseq).

FIG. 8: Proportion of subjects positive or negative for respiratory virus across the five clusters at YZ. The number of virus positive (n=78) and virus negative (n=27) samples are distributed disproportionally across different clusters. Subjects belonging to Haemophilus and Moraxella clusters were all virus positive.

### DETAILED DESCRIPTION OF THE INVENTION

In the following description, certain specific details are set forth in order to provide a thorough understanding of various embodiments. However, one skilled in the art will understand that the embodiments provided may be practiced without these details.

### Certain Definitions

Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

The term “about” when referring to a number or a numerical range means that the number or numerical range referred to is an approximation within experimental variability (or within statistical experimental error), and thus the number or numerical range may vary between 1% and 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

### Microorganisms

The term “microorganism”, as used herein, refers to a living microscopic organism, which may be a single cell or a multicellular organism and which can generally be found in nature. In one embodiment, the microorganism is a bacterium. In another embodiment, the microorganism is a yeast. In another embodiment, the microorganism is a fungus. In some embodiment, the microorganism is an isolated microorganism.

The term “isolated” as used herein means that a material has been removed by the hand of man and exists apart from its original, native environment.

The present disclosure is based on the discovery that the airway microbiome plays a role in asthma pathophysiology. The present disclosure demonstrates that airway microbiota colonization patterns in asthma patients are differentially associated with risk of loss of asthma control and/or severe exacerbations. For example, airway microbiota dominated by Corynebacterium+Dolosigranulum genera are associated with favorable clinical outcomes compared to microbiota dominated by more pathogenic bacteria: Staphylococcus, Streptococcus, and Moraxella.

Microorganisms Associated with Decreased Risk of/Increased Time to Asthma Exacerbations or Loss of Asthma Control

In some embodiments, the present disclosure provides microorganisms associated with a decreased risk of loss of asthma control. In some embodiments, the present disclosure provides microorganisms that are associated with a decreased risk of asthma exacerbations. In some embodiments, the present disclosure provides microorganisms that are associated with an increased time to asthma exacerbations and/or loss of asthma control.

The term “associated with a decreased risk of loss of asthma control”, as used herein, generally means that an elevated or increased amount, concentration or proportion of the microorganism in, e.g., a nasal sample, fecal sample or other biological sample or bodily location, is associated with decreased risk of loss of asthma control. Conversely, a decreased amount, concentration or proportion of the microorganism in, e.g., a nasal sample, fecal sample or other biological sample or bodily location, is associated an increased risk of loss of asthma control.

The term “associated with a decreased risk of asthma exacerbations,” as used herein, generally means that an elevated or increased amount, concentration or proportion of a microorganism in, e.g., a nasal sample, fecal sample or other biological sample or bodily location is associated with a decreased risk of asthma exacerbations. Conversely, a decreased amount, concentration or proportion of a microorganism in, e.g., a nasal sample, fecal sample or other biological sample or bodily location is associated with an increased risk of asthma exacerbations.

The term “associated with an increased time to asthma exacerbations,” as used herein, generally means that an elevated or increased amount, concentration or proportion of a microorganism in, e.g., a nasal sample, fecal sample or other biological sample or bodily location, is associated an increased time to asthma exacerbations, i.e., the individual is protected from asthma exacerbations for a longer period of time. Conversely, a decreased amount, concentration or proportion of a microorganism in, e.g., a nasal sample, fecal sample or other biological sample or bodily location, is associated a decreased time to asthma exacerbations.

In some embodiments, the microorganisms that are associated with a decreased risk of loss of asthma control, a decreased risk of asthma exacerbations, and/or with an increased time to asthma exacerbations and/or loss of asthma control are of the genus Corynebacterium. In some embodiments, the microorganisms are of the genus Dolosigranulum. In some embodiments, the microorganisms are of the genus Corynebacterium and/or Dolosigranulum.

The term “Corynebacterium” refers to a genus of bacteria that are Gram-positive, catalase-positive and aerobic or facultatively anaerobic. Any species and/or subspecies of the Corynebacterium genus is encompassed by the compositions and methods of the present disclosure. Corynebacterium bacteria include lipophilic or non-lipophilic species.

Non-limiting examples of non-lipophilic Corynebacteria include Corynebacterium efficiens; fermentative Corynebacteria such as, but not limited to, Corynebacterium diphtheriae group, Corynebacterium xerosis and Corynebacterium striatum, Corynebacterium minutissimum, Corynebacterium amycolatum, Corynebacterium glucuronolyticum, Corynebacterium argentoratense, Corynebacterium matruchotii, Corynebacterium glutamicum and Corynebacterium sp.; non-fermentative Corynebacteria such as, but not limited to, Corynbacterium afermentas subsp. Afermentans, Corybacterium auris, Corynebacterium pseudodiphtheriticum, and Corynebacterium propinquum. Non-limiting of examples of lipophilic Corynebacteria include Corynebacterium uropygiale, Corynebacterium jeikeium, Corynebacterium urealyticum, Corynebacterium afermentans subsp. Lipophilum, Corynebacterium accolens, Corynebacterium macginleyi, CDC coryneform groups F-1 and G, and Corynebacterium bovis. In other embodiments, the corynebacteria is Corynebacterium kroppenstedtii. In some embodiments, the Corynebacteria is Corynebacterium propinquum. In some embodiments, the Corynebacteria is Corynebacterium pseudodiphtheriticum. In some embodiments, the Corynebacteria is Corynebacterium accolens.

In some embodiments, the microorganisms of genus Corynebacterium comprise microorganisms of Corynebacterium species C. pseudodiphtheriticum or C. accolens. In some embodiments, wherein the microorganism of genus Corynebacterium comprise 16S RNA sequences with 99% identity to any one of SEQ ID NOs. 7-8.

In some embodiments, the microorganisms of genus Dolosigranulum comprise microorganisms of Dolosigranulum species D. pigrum. In some embodiments, the microorganism of genus Dolosigranulum comprise 16S RNA sequences with 99% identity to SEQ ID NO. 9.

Microorganisms Associated with Increased Risk of Asthma Exacerbations or Loss of Asthma Control, and/or with Decreased Time to Loss of Asthma Control.

In some embodiments, the present disclosure provides microorganisms associated with an increased risk of loss of asthma control. In some embodiments, the present disclosure provides microorganisms that are associated with an increased risk of asthma exacerbations. In some embodiments, the present disclosure provides microorganisms that are associated with a decreased time to loss of asthma control.

The term “associated with an increased risk of loss of asthma control”, as used herein, generally means that an elevated or increased amount, concentration or proportion of a microorganism in, e.g., a nasal sample, fecal sample or other biological sample or bodily location, is associated with an increased risk of loss of asthma control. Conversely, a decreased amount, concentration or proportion of the microorganism in, e.g., nasal microbiota, fecal samples or other body components, is associated a decreased risk of loss of asthma control.

The term “associated with an increased risk of asthma exacerbations”, as used herein, generally means that an elevated or increased amount, concentration or proportion of a microorganism in, e.g., a nasal sample, fecal sample or other biological sample or bodily location, is associated with an increased risk of asthma exacerbations. Conversely, a decreased amount, concentration or proportion of the microorganism in, e.g., nasal microbiota, fecal samples or other body components, is associated a decreased risk of asthma exacerbations.

The term “associated with a decreased time to loss of asthma control”, or as used herein, generally means that an elevated or increased amount, concentration or proportion of a microorganism in, e.g., a nasal sample, fecal sample or other biological sample or bodily location, is associated a decreased time to loss of asthma control, i.e., the individual may reach loss of asthma control at a faster rate. Conversely, a decreased amount, concentration or proportion of a microorganism in, e.g., a nasal sample, fecal sample or other biological sample or bodily location, is associated an increased time to loss of asthma control, i.e., the individual is protected from loss of asthma control for a longer period of time.

In some embodiments, the microorganisms that are associated with an increased risk of loss of asthma control, an increased risk of asthma exacerbations, and/or with a decreased time to loss of asthma control are of the genus Moraxella. In some embodiments, the microorganisms are of the genus Staphylococcus. In some embodiments, the microorganisms are of the genus Streptococcus. In some embodiments, the microorganisms are of the genera Moraxella and Streptococcus. In some embodiments, the microorganisms are of the genera Moraxella and Staphylococcus. In some embodiments, the microorganisms are of the genera Streptococcus and Staphylococcus. In some embodiments, the microorganisms are of the genera Moraxella and Streptococcus and Staphylococcus.

The term “Moraxella” refers to a genus of bacteria that are Gram-negative, oxidase-positive, catalase-positive, and are from the family Moraxellaceae. Any species and/or subspecies of the Moraxella genus is encompassed by the compositions and methods of the present disclosure. Non-limiting examples of Moraxella species include M. atlantae, M. boevrei, M. bovis, M. bovoculi, M. canis, M. caprae, M. catarrhalis, M. caviae, M. cuniculi, M. equi, M. lacunata, M. lincolnii, M. nonliquefaciens, M. oblonga, M. osloensis, M. pluranimalium, M. porci, and M. saccharolytica. In some embodiments, the Moraxella species is M. catarrhalis. In some embodiments, the Moraxella species is M. nonliquefaciens. In some embodiments, the Moraxella species is M. lincolnii.

In some embodiments, Moraxella species comprise 16S RNA sequences with 99% identity to any one of SEQ ID NOs. 1-3.

The term “Staphylococcus” refers to a genus of bacteria that are Gram-positive, facultative anerobic bacteria in the family Staphylococcaceae. Any species and/or subspecies of the Staphylococcus genus is encompassed by the compositions and methods of the present disclosure.

Non-limiting examples of Staphylococcus species include Staphylococcus aureus group, e.g., S. argenteus, S. aureus, S. schweitzeri, S. simiae; Staphylococcus auricularis group—e.g., S. auricularis; Staphylococcus carnosus group—S. carnosus, S. condimenti, S. debuckii, S. massiliensis, S. piscifermentans, S. simulans; Staphylococcus epidermidis group—e.g., S. capitis, S. caprae, S. epidermidis, S. saccharolyticus; Staphylococcus haemolyticus group—e.g., S. borealis, S. devriesei, S. haemolyticus, S. hominis; Staphylococcus hyicus-intermedius group—e.g., S. agnetis, S. chromogenes, S. cornubiensis, S. felis, S. delphini, S. hyicus, S. intermedius, S. lutrae, S. microti, S. muscae, S. pseudintermedius, S. rostri, S. schleiftri; Staphylococcus lugdunensis group—e.g., S. lugdunensis; Staphylococcus saprophyticus group—e.g., S. arlettae, S. caeli, S. cohnii, S. equorum, S. gallinarum, S. kloosii, S. leeli, S. nepalensis, S. saprophyticus, S. succinus, S. xylosus; Staphylococcus sciuri group—e.g., S. fleurettii, S. lentus, S. sciuri, S. stepanovicii, S. vitulinus; Staphylococcus simulans group—e.g., S. simulans; and Staphylococcus warneri group—e.g., S. pasteuri, S. warneri. in some embodiments, the Staphylococcus species is S. aureus. In some embodiments, the Staphylococcus species is S. epidermidis.

In some embodiments, the microorganisms of genus Staphylococcus, comprise microorganisms of Staphylococcus species S. aureus or S. epidermidis. In some embodiments, the microorganism of Staphylococcus species comprises 16S RNA sequences with 99% identity to any one of SEQ ID NOs. 4-5.

The term “Streptococcus” refers to a genus of Gram-positive bacteria of the family Streptococcaceae. Any species and/or subspecies of the Streptococcus genus is encompassed by the compositions and methods of the present disclosure.

Non-limiting examples of Streptococcus species include Streptococcus acidominimus, Streptococcus agalactiae, Streptococcus alactolyticus, Streptococcus anginosus, Streptococcus australis, Streptococcus caballi, Streptococcus cameli, Streptococcus canis Streptococcus caprae, Streptococcus castoreus, Streptococcus criceti, Streptococcus constellatus Streptococcus cristatus, Streptococcus cuniculi, Streptococcus danieliae, Streptococcus dentasini, Streptococcus dentiloxodontae, Streptococcus dentirousetti, Streptococcus devriesei, Streptococcus didelphis, Streptococcus downei Streptococcus dysgalactiae, Streptococcus entericus, Streptococcus equi, Streptococcus equinus Streptococcus ferus, Streptococcus gallinaceus, Streptococcus gallolyticus, Streptococcus gordonii, Streptococcus halichoeri, Streptococcus halotolerans, Streptococcus henryi, Streptococcus himalayensis, Streptococcus hongkongensis, Streptococcus hyointestinalis, Streptococcus hyovaginalis, Streptococcus ictaluri, Streptococcus infantarius, Streptococcus infantis, Streptococcus iniae, Streptococcus intermedius, Streptococcus lactarius, Streptococcus loxodontisalivarius, Streptococcus lutetiensis, Streptococcus macacae, Streptococcus marimammalium, Streptococcus marmotae, Streptococcus massiliensis, Streptococcus merionis, Streptococcus minor, Streptococcus mitis, Streptococcus moroccensis, Streptococcus mutans, Streptococcus oralis, Streptococcus oricebi, Streptococcus oriloxodontae, Streptococcus orisasini, Streptococcus orisratti, Streptococcus orisuis, Streptococcus ovis, Streptococcus panodentis, Streptococcus pantholopis, Streptococcus parasanguinis, Streptococcus parasuis, Streptococcus parauberis, Streptococcus peroris, Streptococcus pharyngis, Streptococcus phocae, Streptococcus pluranimalium, Streptococcus plurextorum, Streptococcus pneumoniae, Streptococcus porci, Streptococcus porcinus, Streptococcus porcorum, Streptococcus pseudopneumoniae, Streptococcus pseudoporcinus, Streptococcus pyogenes, Streptococcus ratti, Streptococcus rifensis, Streptococcus rubneri, Streptococcus rupicaprae, Streptococcus salivarius Streptococcus saliviloxodontae, Streptococcus sanguinis, Streptococcus sinensis, Streptococcus sobrinus, Streptococcus suis, Streptococcus tangierensis, Streptococcus thoraltensis, Streptococcus troglodytae, Streptococcus troglodytidis, Streptococcus tigurinus Streptococcus thermophilus Streptococcus uberis, Streptococcus urinalis, Streptococcus ursoris, Streptococcus vestibularis, Streptococcus zooepidemicus, Viridans streptococci, and Streptococcus anginosus group. In some embodiments, the Streptococcus species is Streptococcus pneumoniae. In some embodiments, the Streptococcus species is Streptococcus sanguinis. In some embodiments, the Streptococcus species is Streptococcus mitis. In some embodiments, the Streptococcus species is Streptococcus infantis. In some embodiments, the Streptococcus species is Streptococcus oralis.

In some embodiments, the isolated Streptococcus microorganism comprises microorganisms of Streptococcus species S. pneumoniae. In some embodiments, the microorganisms of Streptococcus species comprise 16S RNA sequences with 99% identity to SEQ ID NO. 6.

In some embodiments, the isolated microorganism is a purified microorganism. As used herein, the term “purified” means can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Diagnostic Methods

In some embodiments, the present disclosure demonstrates the diagnostic utility of measuring nasal microbiome in predicting the risk of asthma exacerbations and/or loss of asthma control in asthma patients. In some embodiments, the composition of the nasal microbiome may be a biomarker for predicting the risk of asthma exacerbations and/or loss of asthma control in asthma patients. In some embodiments, the abundance, amount, concentration or proportion of bacteria in the nasal microbiome and/or a change in the abundance, amount, concentration or proportion of bacteria in the nasal microbiome is predictive of an increased risk of asthma exacerbations. In some embodiments, the abundance, amount, concentration or proportion of bacteria in the nasal microbiome and/or changes in the abundance, amount, concentration or proportion of bacteria in the nasal microbiome is predictive of a decreased or an increased time to asthma exacerbations. In some embodiments, the abundance, amount, concentration or proportion of bacteria in the nasal microbiome and/or changes in the abundance, amount, concentration or proportion of bacteria in the nasal microbiome is predictive of an increased risk of loss of asthma control in the subject. In some embodiments, the asthma patient exhibits early signs of loss of asthma control (e.g., has entered the Yellow Zone). In other embodiments, the asthma patient does not exhibit signs of loss of asthma control.

Thus, in some aspects, provided herein is a method comprising (a) acquiring a biological sample from an upper airway of an individual with early signs of loss of asthma control; (b) identifying a plurality of microorganisms in the biological sample; and (c) determining an amount, concentration, or proportion of at least one microorganism in the biological sample. In some embodiments, the microorganism is selected from the group consisting of microorganisms of genus Moraxella, microorganisms of genus Staphylococcus, microorganisms of genus Streptococcus, microorganisms of genus Corynebacterium, and microorganisms of genus Dolosigranulum. In some embodiments, the microorganism is a species or strain of any one or more of Moraxella, Staphylococcus, Streptococcus, Corynebacterium, or Dolosigranulum, as described herein.

In some aspects, provided herein is a method comprising (a) acquiring a biological sample from an upper airway of an individual with asthma, (b) analyzing a plurality of polynucleotides extracted from the biological sample, and (c) determining an amount, concentration, or proportion of a microorganism in the biological sample, wherein the microorganism is selected from the group consisting of microorganisms of genus Moraxella, microorganisms of genus Staphylococcus, microorganisms of genus Streptococcus microorganisms of genus Corynebacterium, and microorganisms of genus Dolosigranulum. In some embodiments, the microorganism is a species or strain of any one or more of Moraxella, Staphylococcus, Streptococcus, Corynebacterium, or Dolosigranulum, as described herein.

In some embodiments, the step of determining the amount, concentration or proportion of a microorganism comprises determining an amount, concentration, or proportion of two or more microorganisms. In other embodiments, the step of determining the amount, concentration or proportion of a microorganism comprises determining an amount, concentration, or proportion of three or more microorganisms. In other embodiments, the step of determining the amount, concentration or proportion of a microorganism comprises determining an amount, concentration, or proportion of four or more microorganisms. In other embodiments, the step of determining the amount, concentration or proportion of a microorganism comprises determining an amount, concentration, or proportion of five or more microorganisms. In other embodiments, the step of determining the amount, concentration or proportion of a microorganism comprises determining an amount, concentration, or proportion of six or more microorganisms. In other embodiments, the step of determining the amount, concentration or proportion of a microorganism comprises determining an amount, concentration, or proportion of seven or more microorganisms. In other embodiments, the step of determining the amount, concentration or proportion of a microorganism comprises determining an amount, concentration, or proportion of eight or more microorganisms. In other embodiments, the step of determining the amount, concentration or proportion of a microorganism comprises determining an amount, concentration, or proportion of nine or more microorganisms. In other embodiments, the step of determining the amount, concentration or proportion of a microorganism comprises determining an amount, concentration, or proportion of ten or more microorganisms. In other embodiments, the step of determining the amount, concentration or proportion of a microorganism comprises determining an amount, concentration, or proportion of greater than ten or more microorganisms, for example fifteen, twenty, twenty-five, thirty or even more microorganisms.

In some embodiments relating to individuals who demonstrate early signs of loss of asthma control, the step of determining the amount, concentration or proportion of a microorganism comprises determining that an amount, concentration, or proportion of the microorganism in the biological sample is altered relative to a baseline an amount, concentration, or proportion of the microorganism in a biological sample acquired from an individual before the onset of early signs of loss of asthma control.

In some embodiments, the step of determining the amount, concentration or proportion of a microorganism comprises determining that an amount, concentration, or proportion of the microorganism in the biological sample is altered relative to an amount, concentration, or proportion of the microorganism a reference biological sample isolated from an individual who has not been diagnosed with asthma.

In some embodiments, an increased amount, concentration, or proportion of a microorganism of genus Moraxella, Moraxella species, e.g., M. catarrhalis, M. nonliquefaciens or M. lincolnii, genus Staphylococcus, Staphylococcus species, e.g., S. aureus or S. epidermidis, genus Streptococcus, or Streptococcus species, e.g., S. pneumoniae, S. sanguinis, S. mitis, S. infantis or S. oralis is indicative of an increased risk of asthma exacerbations in the subject.

In some embodiments, an increased amount, concentration, or proportion of a microorganism of genus Moraxella, Moraxella species, e.g., M. catarrhalis, M. nonliquefaciens or M. lincolnii, genus Staphylococcus, Staphylococcus species, e.g., S. aureus or S. epidermidis, genus Streptococcus, or Streptococcus species, e.g., S. pneumoniae, S. sanguinis, S. mitis, S. infantis or S. oralis is indicative of a decreased time to loss of asthma control in the subject.

In some embodiments, an increased amount, concentration, or proportion of a microorganism of genus Moraxella, Moraxella species, e.g., M. catarrhalis, M. nonliquefaciens or M. lincolnii, genus Staphylococcus, Staphylococcus species, e.g., S. aureus or S. epidermidis, genus Streptococcus, or Streptococcus species, e.g., S. pneumoniae, S. sanguinis, S. mitis, S. infantis or S. oralis is indicative of an increased risk of loss of asthma control in the subject.

In some embodiments, a decreased amount, concentration, or proportion of a microorganism of genus Corynebacterium, Corynebacterium species, e.g., C. propinquum, C. pseudodiphtheriticum, or C. accolens, genus Dolosigranulum or Dolosigranulum species, e.g., D. pigrum, is indicative of an increased risk of asthma exacerbations in the subject. In some embodiments, a decreased amount, concentration, or proportion of a microorganism of genus Corynebacterium, Corynebacterium species, e.g., C. propinquum, C. pseudodiphtheriticum, or C. accolens, genus Dolosigranulum or Dolosigranulum species, e.g., D. pigrum, is indicative of a decreased time to loss of asthma control in the subject.

In some embodiments, a decreased amount, concentration, or proportion of a microorganism of genus Corynebacterium, Corynebacterium species, e.g., C. propinquum, C. pseudodiphtheriticum, or C. accolens, genus Dolosigranulum or Dolosigranulum species, e.g., D. pigrum, is indicative an increased risk of loss of asthma control in the subject.

In some embodiments, step (b) comprises analyzing a plurality of polynucleotides extracted from the biological sample. In some embodiments, the step of analyzing a plurality of polynucleotides extracted from the biological sample comprises detecting a 16S rRNA sequence. In other embodiments, the step of analyzing a plurality of polynucleotides extracted from the biological sample comprises hybridizing a polynucleotide to an oligonucleotide array. In other embodiments, the step of analyzing a plurality of polynucleotides extracted from the biological sample comprises hybridizing a polynucleotide to an oligonucleotide bait. The oligonucleotide bait may comprise a sequence complementary to a 16S rRNA sequence. In other embodiments, the step of analyzing a plurality of polynucleotides extracted from the biological sample comprises amplification of a segment of a polynucleotide. The segment of the polynucleotide may comprise a 16S rRNA sequence. In other embodiments, the step of analyzing a plurality of polynucleotides extracted from the biological sample comprises sequencing a polynucleotide or a segment of a polynucleotide. The segment of the polynucleotide may comprise any sequence of the bacterial genome.

The term “16S rRNA gene” as used herein, refers to a bacterial gene encoding the component of the 30S small subunit of a prokaryotic ribosome that binds to the Shine-Dalgarno sequence. Sequence analysis of the 16S ribosomal RNA (rRNA) gene has been widely used to identify bacterial taxonomy and perform taxonomic studies. Bacterial 16S rRNA genes generally contain nine hypervariable regions that demonstrate considerable sequence diversity among different bacteria and can be used for taxonomy identification. The term “hypervariable regions of the 16S rRNA gene” refers to said sequences in the 16S ribosomal rRNA gene, that allow identifying a single bacterial genus or differentiating among a limited number of different species within genera. For example, the hypervariable regions of the 16S rRNA gene may allow to identify or differentiate at least one type of bacterium from a mixture of bacteria. The identification of said regions can be effectuated by techniques well known by the person skilled in the art. Non-limiting examples of such techniques are polymerase chain reaction (PCR) amplification of different regions of 16S rRNA, Real Time polymerase chain reaction (RT-PCR), In situ Hybridization (ISH), Northern blot, Micro-array, and the like.

In some embodiments, the microorganism comprises 16SrRNA set forth in Table 1.

TABLE 1

| Species | SEQ ID | Sequence |
|---|---|---|
| Moraxella | 1 | ATTGAACGCTGGCGGCAGGCTTAACACATGCAAGTCGAACGAAGTTAGGA |
| catarrhalis | | AGCTTGCTTCTGATACTTAGTGGCGGACGGGTGAGTAATGCTTAGGAATC |
| | | TGCCTAGTAGTGGGGGATAACTTGGGGAAACCCAAGCTAATACCGCATAC |
| | | GACCTACGGGTGAAAGGGGGCTTTTAGCTCTCGCTATTAGATGAGCCTAA |
| | | GTCGGATTAGCTGGTTGGTGGGGTAAAGGCCTACCAAGGCGACGATCTGT |
| | | AGCTGGTCTGAGAGGATGATCAGCCACACTGGGACTGAGACACGGCCCAG |
| | | ACTCCTACGGGAGGCAGCAGTGGGGAATATTGGACAATGGGCGAAAGCCT |
| | | GATCCAGCCATGCCGCGTGTGTGAAGAAGGCCTTTTGGTTGTAAAGCACT |
| | | TTAAGTGGGGAGGAAAAGCTTATGGTTAATACCCATAAGCCCTGACGTTA |
| | | CCCACAGAATAAGCACCGGCTAACTCTGTG |
| Moraxella | 2 | ATTGAACGCTGGCGGCAGGCTTAACACATGCAAGTCGAACGATGAAGTCT |
| nonliquefaciens | | AGCTTGCTAGACGGATTAGTGGCGAACGGGTGAGTAATGCTTAGGAATCT |
| | | GCCTATTAGTGGGGGATAACGTAGGGAAACTTACGCTAATACCGCATACG |
| | | ACCTACGGGTGAAAGGGGGCGTTTAGCTCTCGCTAATAGATGAGCCTAAG |
| | | TCGGATTAGCTAGTTGGTGGGGTAAAGGCCTACCAAGGCGACGATCTGTA |
| | | GCTGGTCTGAGAGGATGATCAGCCACACTGGGACTGAGACACGGCCCAGA |
| | | CTCCTACGGGAGGCAGCAGTGGGGAATATTGGACAATGGGCGAAAGCCTG |
| | | ATCCAGCCATGCCGCGTGTGTGAAGAAGGCCTTTTGGTTGTAAAGCACTT |
| | | TAAGTGGGGAGGAAAAGCTTGTGGTTAATACCCATAAGCCCTGACGTTAC |
| | | CCACAGAATAAGCACCGGCTAACTCTGTG |
| Moraxella lincolnii | 3 | ATTGAACGCTGGCGGCAGGCTTAACACATGCAAGTCGAACGAAGAGGTCT |
| | | AGCTTGCTAGACTGATTAGTGGCGAACGGGTGAGTAACATTTAGGAATCT |
| | | ACCTTATAGAGGGGGATAGCTCGGGGAAACTCGAATTAATACCGCATACG |
| | | ACCTACGGGTGAAAGGGGGCGCAAGCTCTTGCTATAAGATGAGCCTAAAC |
| | | CAGATTAGCTAGTTGGTGGGGTAAAGGCCTACCAAGGCGACGATCTGTAG |
| | | CTGGTCTGAGAGGATGATCAGCCACACTGGGACTGAGACACGGCCCAGAC |
| | | TCCTACGGGAGGCAGCAGTGGGGAATATTGGACAATGGGCGAAAGCCTGA |
| | | TCCAGCCATGCCGCGTGTGTGAAGAAGGCCTTATGGTTGTAAAGCACTTT |
| | | AAGTGGGGAGGAAAAGCTTATGGATAACACCCATAGGTTCTGACGTTACC |
| | | CACAGAATAAGCACCGGCTAACTCTGTG |
| Staphylococcus | 4 | GATGAACGCTGGCGGCGTGCCTAATACATGCAAGTCGAGCGAACGGACGA |
| aureus | | GAAGCTTGCTTCTCTGATGTTAGCGGCGGACGGGTGAGTAACACGTGGAT |
| | | AACCTACCTATAAGACTGGGATAACTTCGGGAAACCGGAGCTAATACCGG |
| | | ATAATATTTTGAACCGCATGGTTCAAAAGTGAAAGACGGTCTTGCTGTCA |
| | | CTTATAGATGGATCCGCGCTGCATTAGCTAGTTGGTAAGGTAACGGCTTA |
| | | CCAAGGCAACGATGCATAGCCGACCTGAGAGGGTGATCGGCCACACTGGA |
| | | ACTGAGACACGGTCCAGACTCCTACGGGAGGCAGCAGTAGGGAATCTTCC |
| | | GCAATGGGCGAAAGCCTGACGGAGCAACGCCGCGTGAGTGATGAAGGTCT |
| | | TCGGATCGTAAAACTCTGTTATTAGGGAAGAACATATGTGTAAGTAACTG |
| | | TGCACATCTTGACGGTACCTAATCAGAAAGCCACGGCTAACTACGTG |
| Staphylococcus | 5 | GATGAACGCTGGCGGCGTGCCTAATACATGCAAGTCGAGCGAACAGATGA |
| epidermidis | | GGAGCTTGCTCCTCTGACGTTAGCGGCGGACGGGTGAGTAACACGTGGAT |
| | | AACCTACCTATAAGACTGGGATAACTTCGGGAAACCGGAGCTAATACCGG |
| | | ATAATATATTGAACCGCATGGTTCAATAGTGAAAGACGGTTTTGCTGTCA |
| | | CTTATAGATGGATCCGCGCCGCATTAGCTAGTTGGTAAGGTAACGGCTTA |
| | | CCAAGGCGACGATGCGTAGCCGACCTGAGAGGGTGATCGGCCACACTGGA |
| | | ACTGAGACACGGTCCAGACTCCTACGGGAGGCAGCAGTAGGGAATCTTCC |
| | | GCAATGGGCGAAAGCCTGACGGAGCAACGCCGCGTGAGTGATGAAGGTCT |
| | | TCGGATTGTAAAACTCTGTTATTAGGGAAGAACAAATGTGTAAGTAACTA |
| | | TGCACGTCTTGACGGTACCTAATCAGAAAGCCACGGCTAACTACGTG |
| Streptococcus | 6 | GACGAACGCTGGCGGCGTGCCTAATACATGCAAGTAGAACGCTGAAGGAG |
| pneumoniae | | GAGCTTGCTTCTCTGGATGAGTTGCGAACGGGTGAGTAACGCGTAGGTAA |
| | | CCTGCCTGGTAGCGGGGGATAACTATTGGAAACGATAGCTAATACCGCAT |
| | | AAGAGTAGATGTTGCATGACATTTGCTTAAAAGGTGCACTTGCATCACTA |
| | | CCAGATGGACCTGCGTTGTATTAGCTAGTTGGTGGGGTAACGGCTCACCA |
| | | AGGCGACGATACATAGCCGACCTGAGAGGGTGATCGGCCACACTGGGACT |
| | | GAGACACGGCCCAGACTCCTACGGGAGGCAGCAGTAGGGAATCTTCGGCA |
| | | ATGGACGGAAGTCTGACCGAGCAACGCCGCGTGAGTGAAGAAGGTTTTCG |
| | | GATCGTAAAGCTCTGTTGTAAGAGAAGAACGAGTGTGAGAGTGGAAAGTT |
| | | CACACTGTGACGGTATCTTACCAGAAAGGGACGGCTAACTACGTG |
| Corynebacterium | 7 | GACGAACGCTGGCGGCGTGCTTAACACATGCAAGTCGAACGGAAAGGCCT |
| pseudodiphtheriticum | | CTTCGGAGGTACTCGAGTGGCGAACGGGTGAGTAACACGTGGGTGATCTG |
| | | CCCTGCACTCTGGGATAAGCCTGGGAAACTGGGTCTAATACCGGATAGGA |
| | | CCATGCTTTAGTGTGTGTGGTGGAAAGTTTTTTCGGTGTAGGATGAGCCC |
| | | GCGGCCTATCAGCTTGTTGGTGGGGTAATGGCCTACCAAGGCGGCGACGG |
| | | GTAGCCGGACTGAGAGGTTGGTCGGCCACATTGGGACTGAGATACGGCCC |
| | | AGACTCCTACGGGAGGCAGCAGTGGGGAATATTGCACAATGGGCGCAAGC |
| | | CTGATGCAGCGACGCCGCGTGGGGGATGACGGCCTTCGGGTTGTAAACTC |
| | | CTTTCGCCAGGGACGAAGCGTTTTGTGACGGTACCTGGAGAAGAAGCACC |
| | | GGCTAACTACGTG |
| Corynebacterium | 8 | GACGAACGCTGGCGGCGTGCTTAACACATGCAAGTCGAACGGAAAGGCCC |
| accolens | | TGCTTGCAGGGTACTCGAGTGGCGAACGGGTGAGTAACACGTGGGTGATC |
| | | TGCCCTGCACTTCGGGATAAGCTTGGGAAACTGGGTCTAATACCGGATAG |
| | | GAGCCATCTTTAGTGTGGTGGTTGGAAAGTTTTTTCGGTGTAGGATGAGC |
| | | TCGCGGCCTATCAGCTTGTTGGTGGGGTAATGGCCTACCAAGGCGGCGAC |
| | | GGGTAGCCGGCCTGAGAGGGTGTACGGCCACATTGGGACTGAGATACGGC |
| | | CCAGACTCCTACGGGAGGCAGCAGTGGGGAATATTGCACAATGGGCGCAA |
| | | GCCTGATGCAGCGACGCCGCGTGGGGGATGAAGGCCTTCGGGTTGTAAAC |
| | | TCCTTTCGCTAGGGACGAAGCTTTTTGTGACGGTACCTAGATAAGAAGCA |
| | | CCGGCTAACTACGTG |
| Dolosigranulum | 9 | GACGAACGCTGGCGGCATGCCTAATACATGCAAGTCGAACGATGATATCA |
| pigrum | | CTGCTTGCAGTGATTGATTAGTGGCGAACGGGTGAGTAACACGTGAGGAA |
| | | CTTGCCCATGAGCGGGGGACAACATTCGGAAACGGATGCTAATACCCCAT |
| | | AGGTGGATTGGTCGCATGACGAATTCATTAAAGGTGGCTTTGCTACCACT |
| | | CATGGATAGCCTCGCGGCGTATTAGCTAGTTGGTAAGGTAATGGCTTACC |
| | | AAGGCAGTGATACGTAGCCGACTTGAGAGGGTGATCGGCCACACTGGGAC |
| | | TGAGACACGGCCCAGACTCCTACGGGAGGCAGCAGTAGGGAATCTTCCAC |
| | | AATGGGTGCAAACCTGATGGAGCAATGCCGCGTGAGTGAAGAAGGTCTTC |
| | | GGATCGTAAAGCTCTGTTGTTAGAGAAGAACACGTGCTAGGTAACTACTA |
| | | GCGCCTTGACGGTATCTAACCAGAAAGTCACGGCTAACTACGTG |

In some embodiments, step (b) comprises analyzing a plurality of polypeptides extracted from the biological sample. In some embodiments, analyzing a plurality of polypeptides extracted from the biological sample comprises matrix desorption/ionization time of flight mass spectrometry (MALDI-TOF), as known in the art.

In other embodiments, step (b) comprises performing Whole Genome Sequencing (WGS) as known in the art.

In some embodiments, the step of determining the amount, concentration or proportion of a microorganism comprises determining that a sequence of a polynucleotide has at least about 80% identity to a reference sequence of the microorganism. In some embodiments, the step of determining the amount, concentration or proportion of a microorganism comprises determining that a sequence of a polynucleotide has at least about 85% identity to a reference sequence of the microorganism. In some embodiments, the step of determining the amount, concentration or proportion of a microorganism comprises determining that a sequence of a polynucleotide has at least about 90% identity to a reference sequence of the microorganism. In some embodiments, the step of determining the amount, concentration or proportion of a microorganism comprises determining that a sequence of a polynucleotide has at least about 95% identity to a reference sequence of the microorganism. In some embodiments, the step of determining the amount, concentration or proportion of a microorganism comprises determining that a sequence of a polynucleotide has at least about 96% identity to a reference sequence of the microorganism. In some embodiments, the step of determining the amount, concentration or proportion of a microorganism comprises determining that a sequence of a polynucleotide has at least about 97% identity to a reference sequence of the microorganism. In some embodiments, the step of determining the amount, concentration or proportion of a microorganism comprises determining that a sequence of a polynucleotide has at least about 98% identity to a reference sequence of the microorganism. In some embodiments, the step of determining the amount, concentration or proportion of a microorganism comprises determining that a sequence of a polynucleotide has at least about 99% identity to a reference sequence of the microorganism.

In some embodiments, the sequence of a polynucleotide may of different bacterial may be compared utilizing differential expression analysis by analyzing a 16S rRNA sequence with a DESeq2 algorithm.

In some embodiments, the biological sample is a nasal blow sample. In some embodiments, the biological nasal blow sample is collected using a nasal swab. In some embodiments, the biological sample is collected from the surface of a respiratory mucosa without a nasopharyngeal cavity using a nasal swab.

In some embodiments, the methods further comprise determining a presence or absence of a respiratory virus in the biological sample. The term “respiratory virus” as used herein encompasses any known or currently unknown respiratory virus. Non-limiting examples of respiratory viruses include enterovirus, rhinovirus, e.g., rhinovirus C, metapneumovirus, coronavirus, respiratory syncytial virus (RSV), adenovirus, bocavirus, rhinovirus, influenza virus, severe acute respiratory syndrome (SARS), SARS-CoV-2 (COVID-19), Middle East Respiratory Syndrome (MERS), or porcine reproductive or respiratory syndrome virus (PRRSV) infection. In some embodiments, the respiratory virus is an enterovirus. In other embodiments, the respiratory virus is a rhinovirus, e.g., rhinovirus C.

In some embodiments, the presence or absence of a respiratory virus is determined by a multiplex polymerase chain reaction (PCR). Additional techniques suitable for detecting the presence or absence of a respiratory virus are Real Time polymerase chain reaction (RT-PCR), In situ Hybridization (ISH), Northern blot, Micro-array, and the like.

In some embodiments, the methods further comprising the step of determining if the individual lives in a household with a pet, e.g., dogs, cats, rabbits, ferrets, pigs, rodents such as gerbils, hamsters, chinchillas, guinea pigs; avian pets such as parrots, passerines or fowls; reptile pets such as turtles; or aquatic pets such as fish or frogs.

In some embodiments, the methods further comprise the step of determining a respiratory rate of the individual, a pulse rate of the individual, or a partial pressure of oxygen in arterial blood of the individual. Methods of determining respiratory rates include, e.g., contact-based methods or contactless methods. For example, respiratory rate may be measured by counting the number of breaths for one minute by counting how many times the chest rises. Alternatively, a fiber-optic breath rate sensor can be used for monitoring patients during a magnetic resonance imaging scan. Various other methods to measure respiratory rate may be used, including impedance pneumography, capnography, as well as wearable sensors such as electrocardiogram, photoplethysmogram or accelerometry signals. Blood oxygen levels may be measured with a pulse oximeter as known in the art. In some embodiments, the methods further comprise a step of measuring a respiratory flow rate. A respiratory flow rate may be measured by any means known in the art, including a peak flow meter.

### Therapeutic Uses

In some embodiments, the present disclosure relates to therapeutic methods for treating conditions and diseases. In some embodiments, the present disclosure relates to therapeutic methods for treating asthma. In some embodiments, the present disclosure relates to methods to treating asthma in an asthma patient. In some embodiments, the present disclosure relates to methods to treating asthma in an asthma patient who exhibits early signs of loss of asthma control. In some embodiments, the present disclosure relates to methods to treating or controlling asthma exacerbations in an asthma patient who exhibits early signs of loss of asthma control. In some embodiments, the present disclosure relates to methods to treating or controlling asthma exacerbations in an asthma patient who is asymptomatic. In some embodiments, the present disclosure relates to methods to preventing or attenuating loss of asthma control in an asthma patient who exhibits early signs of loss of asthma control. The methods comprise administering to the individual an effective amount of an agent that is effective to prevent or attenuate asthma exacerbations.

The term “effective amount” refers to the amount of a therapy which is sufficient to accomplish a stated purpose or otherwise achieve the effect for which it is administered. An effective amount can be sufficient to reduce and/or ameliorate the progression, development, recurrence, severity and/or duration of a given disease, disorder or condition and/or a symptom related thereto. An effective amount can be a “therapeutically effective amount” which refers to an amount sufficient to provide a therapeutic benefit such as, for example, the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. A therapeutically effective amount of a composition described herein can enhance the therapeutic efficacy of another therapeutic agent.

The term “regimen” refers to a protocol for dosing and timing the administration of one or more therapies (e.g., probiotic compositions) for treating a disease, disorder, or condition described herein. A regimen can include periods of active administration and periods of rest as known in the art. Active administration periods include administration of compositions described herein and the duration of time of efficacy of such compositions. Rest periods of regimens described herein include a period of time in which no compound is actively administered, and in certain instances, includes time periods where the efficacy of such compounds can be minimal.

The terms “therapies” and “therapy” refer to any protocol(s), method(s), and/or agent(s) that can be used in the prevention, treatment, management, and/or amelioration of a disease, disorder, or condition or one or more symptoms thereof. In certain instances the term refers to active agents such as a probiotic formulation, a bacterial extract, a prebiotic formulation, an antibiotic agent, an antibacterial agent, or an antimicrobial agent, in each case as described herein. The terms “therapy” and “therapy” can refer to therapies useful in treatment, management, prevention, or amelioration of a disease, disorder, or condition or one or more symptoms thereof known to one skilled in the art, for example, a medical professional such as a physician.

As used herein, when an individual is in a “Green Zone”, the individual does not experience any meaningful asthma symptoms: neither early symptoms of loss of asthma control (“Yellow Zone”), nor asthma exacerbation (“Red Zone”). As used herein, when an individual is a “Yellow Zone”, the individual uses: (a) two or more doses of rescue albuterol in 6 hours; (b) four or more inhalations of rescue albuterol in 6 hours; (c) three or more doses of rescue albuterol in 24 hours; (d) six or more inhalations of rescue albuterol in 24 hours; or (e) the individual stay awake at night due to asthma symptoms that was treated with albuterol.

As used herein, when an individual is in a “Red Zone”, the individual is experiencing asthma exacerbations. For example, an individual is in a Red Zone if the individual coughs or wheezes throughout the day. For example, an individual is in a Red Zone if the individual is short of reach at rest or with talking or walking. an individual is in a Red Zone if the individual's chest sinks in around the ribs or at the neck. An individual is in a Red Zone if the individual uses rescue albuterol or other asthma medication several times a day without adequate response. The Red Zone indicates severe asthma symptoms including shortness of breath while walking, talking, or at rest or use of the chest muscles to breathe. A person in the Red Zone may have wheezing, but wheezing may stop when the amount of air moving through the bronchial tubes becomes dangerously low. In this case, no wheezing is worse than hearing wheezing. In some embodiments, the individual has Peak expiratory flow (PEV) of less than 50% of the person's best peak flow measurement.

In some aspects, the present disclosure generally relates to treating asthma patients, by (a) determining nasal biome make-up so as to identify individuals with an elevated risk for asthma exacerbations and/or loss of asthma control, and (b) administering to such individuals intervention therapy to treat asthma. In some embodiments, probiotic compositions comprising one or more bacteria that are associated with decreased risk of loss of asthma control and/or decreased risk of asthma exacerbations may be administered to an individual with asthma. In some embodiments, probiotic compositions comprising one or more bacteria that are associated with decreased risk of loss of asthma control and/or decreased risk of asthma exacerbations may be administered to an individual with asthma that exhibits early signs of loss of asthma control. In some embodiments, probiotic compositions comprising one or more bacteria that are associated with decreased risk of loss of asthma control and/or decreased risk of asthma exacerbations may be administered to an individual with asthma who is experiencing asthma exacerbation.

In some embodiments, the methods of the present disclosure prevent or attenuate progression of the individual with asthma from a Green Zone to a Yellow Zone. In some embodiments, the methods of the present disclosure prevent or attenuate progression of the individual with asthma from a Green zone to a Red zone. In some embodiments, the methods of the present disclosure prevent or attenuate progression of the individual with asthma from a Yellow Zone to a Red Zone.

In some embodiments, the methods of the present disclosure reduce the time (e.g., hours, days) an individual with asthma in a Yellow Zone. In some embodiments, the methods of the present disclosure reduce the time an individual with asthma in a Yellow Zone and promote transition of the individual from a Yellow Zone to a Green Zone. In some embodiments, the methods of the present disclosure reduce the time an individual with asthma in a Red Zone. In some embodiments, the methods of the present disclosure reduce the time an individual with asthma in a Yellow Zone and promote transition of the individual from a Yellow Zone to a Green Zone.

Thus, in some aspects, provided herein is a method of treating an individual with early signs of loss of asthma control comprising (a) detecting an altered amount, concentration, or proportion of a microorganism in a biological sample from an upper airway of the individual; and (b) administering to the individual an agent that is effective to prevent or attenuate asthma exacerbations. In some embodiments, the detected microorganism is selected from the group consisting of microorganisms of genus Moraxella, microorganisms of genus Staphylococcus, microorganisms of genus Streptococcus microorganisms of genus Corynebacterium, and microorganisms of genus Dolosigranulum. In some embodiments, the microorganism is a species or strain of any one or more of Moraxella, Staphylococcus, Streptococcus, Corynebacterium, or Dolosigranulum, as described herein.

In some embodiments, the early signs of loss of asthma control comprise increased use of asthma intervention therapy or increased asthma symptoms. In other embodiments, the individual with early signs of loss of asthma control used at least one dose of albuterol and remained awake for one night. In other embodiments, the individual with early signs of loss of asthma control used at least two doses of albuterol in 6 hours. In other embodiments, the individual with early signs of loss of asthma control used at least three doses of albuterol in 24 hours.

In other aspects provided herein is a method of treating an individual with asthma comprising (a) detecting an increased amount, concentration, or proportion of a microorganism of genus Moraxella, genus Staphylococcus or genus Streptococcus compared to an amount, concentration, or proportion of a microorganism of genus Corynebacterium or genus Dolosigranulum in a biological sample from an upper airway of the individual; and (b) administering to the individual an agent that is effective to prevent or attenuate asthma exacerbations. In some embodiments, the detected microorganism is a species or strain of any one or more of Moraxella, Staphylococcus, Streptococcus, Corynebacterium, or Dolosigranulum, as described herein.

In some embodiments, the step of detecting comprises (a) acquiring a biological sample from an upper airway of an individual with early signs of loss of asthma control, (b) analyzing a plurality of polynucleotides extracted from the biological sample, and (c) determining an amount, concentration, or proportion of a microorganism in the biological sample. In some embodiments, the microorganism is selected from the group consisting of microorganisms of genus Moraxella, genus Staphylococcus, microorganisms of genus Streptococcus, microorganisms of genus Corynebacterium, and microorganisms of genus Dolosigranulum. In other embodiments, the microorganism is selected from the group consisting of microorganisms of Moraxella species M. catarrhalis, M. nonliquefaciens or M. lincolnii microorganisms of genus Staphylococcus, microorganisms of genus Streptococcus microorganisms of genus Corynebacterium, and microorganisms of genus Dolosigranulum. In some embodiments, the microorganism is a species or strain of any one or more of Moraxella, Staphylococcus, Streptococcus, Corynebacterium, or Dolosigranulum, as described herein.

In some embodiments, the agent is an antibiotic composition as described herein. In other embodiments, the agent is a prebiotic composition as described herein. In other embodiments, the agent is a probiotic composition as described herein. In some embodiments, the probiotic composition comprises an isolated microorganism of genus Corynebacterium, genus Dolosigranulum, or a combination thereof. In some embodiments, the isolated microorganism is a purified microorganism. In some embodiments, the microorganisms of genus Corynebacterium comprise microorganisms of Corynebacterium species C. pseudodiphtheriticum or C. accolens. In some embodiments, wherein the microorganism of genus Corynebacterium comprise 16S RNA sequences with 99% identity to any one of SEQ ID NOs. 7-8. In some embodiments, the microorganisms of genus Dolosigranulum comprise microorganisms of Dolosigranulum species D. pigrum. In some embodiments, the microorganism of genus Dolosigranulum comprise 16S RNA sequences with 99% identity to SEQ ID NO. 9.

In some embodiments, the method further comprises a step of administering a second agent that prevents or attenuates asthma exacerbations. The second agent may be a drug, a prebiotic, a bacterial extract or a fungus.

In some embodiment, the method further comprises a step of determining the individual has a respiratory rate of greater than 60 breaths per minute, a pulse of greater than 120 beats per minute, or a partial pressure of oxygen in arterial blood of less than 60 mM Hg. In some embodiments, the methods of determining respiratory rate precedes the step of administering.

In other aspects, provided herein is a method of modifying a nasal microbiota of an individual comprising administering to the individual a probiotic composition comprising an isolated microorganism at a dose sufficient to cause an increase in an abundance or proportion of the microorganism in the nasal microbiota of the individual, wherein the isolated microorganism is a microorganism of genus Corynebacterium or a microorganism of genus Dolosigranulum, or any species or strain of the foregoing. In some embodiments, the nasal microbiota of the individual comprises an elevated abundance or proportion relative to a reference sample of one or more microorganisms of genus Moraxella, genus Staphylococcus or genus Streptococcus, or a reduced abundance or proportion relative to the reference sample of one or more microorganisms of genus Corynebacterium or genus Dolosigranulum, or any species or strain of the foregoing.

In other aspects, provided herein is a method of treating an individual with a nasal microbiome characterized by an elevated abundance or proportion relative to a reference sample of one or more microorganisms of genus Moraxella, genus Staphylococcus or genus Streptococcus; or a reduced abundance or proportion relative to the reference sample of one or more microorganisms of genus Corynebacterium or genus Dolosigranulum, the method comprising administering to the individual a probiotic composition comprising an isolated microorganism, wherein the isolated microorganism is a microorganism of genus Corynebacterium or genus Dolosigranulum, thereby preventing, delaying or attenuating an asthma exacerbation or loss of asthma control of the individual. In some embodiments, the microorganism is a species or strain of any one or more of Moraxella, Staphylococcus, Streptococcus, Corynebacterium, or Dolosigranulum, as described herein.

In other aspects, provided herein is a method of treating an individual with a nasal microbiome characterized by an elevated abundance or proportion relative to a reference sample of one or more microorganisms of genus Moraxella, genus Staphylococcus or genus Streptococcus; or a reduced abundance or proportion relative to the reference sample of one or more microorganisms of genus Corynebacterium or genus Dolosigranulum, the method comprising administering to the individual a probiotic composition comprising an isolated microorganism, wherein the isolated microorganism is a microorganism of genus Corynebacterium or genus Dolosigranulum, thereby preventing or treating an upper respiratory tract infection of the individual. In some embodiments, the microorganism is a species or strain of any one or more of Moraxella, Staphylococcus, Streptococcus, Corynebacterium, or Dolosigranulum, as described herein.

In some embodiments, the reference sample is a biological sample obtained from an upper airway of the individual before the onset of early signs of loss of asthma control. In other embodiments, the reference sample is a biological sample obtained from an upper airway of an individual not diagnosed with asthma. In other embodiments, the one or more microorganisms of genus Moraxella, genus Staphylococcus or genus Streptococcus comprises one or more microorganisms having comprise 16S RNA sequences with 99% identity to any one of the species in Table 1.

In some embodiments, any of the aforementioned methods may further comprise the step of administering a corticosteroid to an airway of the subject. In some embodiments, the corticosteroid is selected from the group consisting of a class B corticosteroid, a glucocorticosteroid, budesonide, ciclesonide, fluticasone furoate, mometasone furoate, fluticasone propionate, or beclomethasone dipropionate. These, and other, corticosteroids are described, for example, in Swartz, et al., “Corticosteroids: clinical pharmacology and therapeutic use.” Drugs 16(3) (1978), the content of which is hereby incorporated by reference.

### Subjects

Subjects can be, for example, elderly adults, adults, adolescents, pre-adolescents, children, toddlers, infants, neonates, and non-human animals. In some embodiments, a subject is a patient.

In some embodiments, subject or individual treated by the methods of the present disclosure is an asthma patient. In some embodiments, the present disclosure relates to methods to treating asthma in an asthma patient. In some embodiments, the present disclosure relates to methods to treating asthma in an asthma patient who exhibits early signs of loss of asthma control. In some embodiments, the present disclosure relates to methods to treating asthma in an asthma patient who is asymptomatic.

In some embodiments, the subject or individual is a child. In some embodiments, the subject or individual is a child that is diagnosed with asthma. In other embodiments, the child is between about 2 and about 17 years old. In other embodiments, the child is between about 2 and about 16 years old. In other embodiments, the child is between about 2 and about 15 years old. In other embodiments, the child is between about 2 and about 14 years old. In other embodiments, the child is between about 2 and about 13 years old. In other embodiments, the child is between about 2 and about 12 years old. In other embodiments, the child is between about 2 and about 11 years old. In other embodiments, the child is between about 2 and about 17 years old. In other embodiments, the child is between about 2 and about 10 years old. In other embodiments, the child is between about 5 and about 17 years old. In other embodiments, the child is between about 6 and about 17 years old. In other embodiments, the child is between about 7 and about 17 years old. In other embodiments, the child is between about 8 and about 17 years old. In other embodiments, the child is between about 9 and about 17 years old. In other embodiments, the child is between about 10 and about 17 years old. In other embodiments, the child is between about 5 and about 11 years old. In other embodiments, the child is between about 5 and about 10 years old. In other embodiments, the child is between about 5 and about 9 years old. In other embodiments, the child is between about 5 and about 8 years old. In other embodiments, the child is between about 5 and about 7 years old.

In some embodiments, the child is an infant. In other embodiments, the child is less than about 6 months old. In other embodiments, the child is less than about 1 year old. In other embodiments, the child is about 1 year old. In other embodiments, the child is about 2 years old. In other embodiments, the child is about 3 years old. In other embodiments, the child is about 4 years old. In other embodiments, the child is about 5 years old. In other embodiments, the child is about 6 years old. In other embodiments, the child is about 7 years old. In other embodiments, the child is about 8 years old. In other embodiments, the child is about 9 years old. In other embodiments, the child is about 10 years old. In other embodiments, the child is about 11 years old. In other embodiments, the child is about 10 years old. In other embodiments, the child is about 12 years old. In other embodiments, the child is about 13 years old. In other embodiments, the child is about 14 years old. In other embodiments, the child is about 15 years old. In other embodiments, the child is about 16 years old. In other embodiments, the child is about 17 years old.

In some embodiments, the subject or individual treated by the methods of the present disclosure is an adult. In some embodiments, the subject is between about 18 and about 90 years old. In some embodiments, the subject is between about 18 and about 80 years old. In some embodiments, the subject is between about 18 and about 70 years old. In some embodiments, the subject is between about 18 and about 60 years old. In some embodiments, the subject is between about 18 and about 50 years old. In some embodiments, the subject is between about 18 and about 50 years old. In some embodiments, the subject is between about 18 and about 40 years old. In some embodiments, the subject is between about 18 and about 30 years old. In some embodiments, the subject is between about 20 and about 25 years old. In some embodiments, the subject is between about 25 and about 35 years old. In some embodiments, the subject is between about 35 and about 45 years old. In some embodiments, the subject is between about 45 and about 55 years old.

In some embodiments, the subject is about 20 years old, about 25 years old, about 30 years old, about 40 years old, about 45 years old, about 50 years old, about 55 years old, about 60 years old, about 65 years old, about 70 years old, about 75 years old, about 80 years old, about 85 years old, about 90 years old, or older than 90 years old.

### Bacterial Extracts

In some aspects, the present disclosure relates to a bacterial extract for administration to an upper airway of an individual with asthma, wherein the bacterial extract is (a) isolated from a microorganism of genus Corynebacterium or genus Dolosigranulum, and (b) capable of inhibiting growth and/or colonization of the upper airway of the individual by a microorganism of genus Staphylococcus, genus Streptococcus, or genus Moraxella.

The term “bacterial extract” as used herein refers to a bacterial cell lysate or a fraction thereof wherein the cellular extract is able to synthesize a protein from a nucleic acid template without adding other components. In some embodiments, the bacterial extract contains an energy source, such as ATP, GTP and the like. A bacterial extract may be a portion of a lysate from which other cellular components of the lysate have been separated by centrifugation, filtration, selective precipitation, selective immunoprecipitation, chromatography, or other methods. The term “bacterial extract” also includes lysates or fractions thereof that contain exogenous material such as preservatives, stabilizers and reagents that enhance cell free protein synthesis (CFPS). The bacterial extract may be derived directly from lysed bacteria, from purified components or combinations of both.

In some embodiment, the bacterial extract is selected from a cell supernatant, cell debris, cell walls, and a DNA extract. In some embodiments, the bacterial extract comprises a glycan.

In some embodiments, the extract may be obtained using any method suitable for lysing bacteria. For example, the extract may be obtained by lysing bacterial cells under basic conditions (e.g., by means of an organic or inorganic base). Mechanical methods of lysing bacterial cells may be used too, such as vortex. If required, the extract may be filtered by passage through one or more microfilters or ultrafilters before use, for example in order to remove cell wall fragments from the bacterial extract. The extract may be in liquid or solid form. The extract may be lyophilized and conditioned in powder form.

In some embodiments, the microorganism of genus Corynebacterium or genus Dolosigranulum in the bacterial extract is selected from the group consisting comprise 16S RNA sequences with 99% identity to any one SEQ. ID. 7-9. In other embodiments, the bacterial extract is secreted by a microorganism of genus Corynebacterium. In other embodiments, the bacterial extract is secreted by a microorganism of genus Dolosigranulum.

In some embodiments, wherein the bacterial extract is isolated from an outer surface of the microorganism of genus Corynebacterium or genus Dolosigranulum.

In some aspects, provided herein is a probiotic formulation comprising the bacterial extract of the present disclosure, and a pharmaceutically acceptable carrier.

### Probiotic and Prebiotic Formulations

In some aspects, provided herein is a pharmaceutical composition or formulation for preventing, delaying or attenuating asthma exacerbations or loss of asthma control comprising a pharmaceutically acceptable unit dose of a composition comprising one or more isolated microorganisms of genus Corynebacterium or genus Dolosigranulum, or a combination thereof, or species or strains of the foregoing, and a pharmaceutically acceptable carrier.

In some embodiments, the pharmaceutical composition is a probiotic composition. As used herein, the term “probiotic” live microorganisms that, when administered in adequate amounts, confer a health benefit on the host.

In some embodiments, the pharmaceutical composition is a prebiotic composition. The term “prebiotic” as used herein generally refers to ingredients that can affect the growth and/or activity of microorganisms in a subject or host (e.g., can allow for changes in the composition and/or activity in the microbiome) and/or can confer a health benefit on the subject).

In some embodiments, the present disclosure provides probiotic compositions for treating asthma. In some embodiments, the probiotic compositions comprise microorganisms that are associated with decreased risk of loss of asthma control. In some embodiments, the probiotic compositions comprise microorganisms that are associated with decreased risk of asthma exacerbations. In some embodiments, the probiotic compositions comprise microorganisms of the genus Corynebacterium. In some embodiments, the probiotic compositions comprise microorganisms of the genus Dolosigranulum. In some embodiments, the probiotic compositions comprise microorganisms of genera Corynebacterium and Dolosigranulum. In some embodiments, the probiotic compositions comprise microorganisms of one or more Corynebacterium species. In some embodiments, the probiotic compositions comprise microorganisms of one or more Dolosigranulum species. In some embodiments, the probiotic compositions comprise microorganisms of one or more species of Corynebacterium and Dolosigranulum.

Prebiotic compounds that may be incorporated into the probiotic or prebiotic formulations include, but are not limited to, a non-digestible carbohydrate such as an oligo- or polysaccharide, or a sugar alcohol, which is not degraded or absorbed in the upper digestive tract. Examples of prebiotics include, but are not limited to, complex carbohydrates, complex sugars, resistant dextrins, resistant starch, amino acids, peptides, nutritional compounds, biotin, polydextrose, fructooligosaccharide (FOS), galactooligosaccharides (GOS), inulin, lignin, psyllium, chitin, chitosan, gums (e.g. guar gum), high amylose cornstarch (HAS), cellulose, beta-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, and xylooligosaccharides (XOS). Prebiotic substrates, such as these, improve the colonization and survival of the bacteria in vivo.

In some embodiments, the methods further include administering a second probiotic composition. Any probiotic composition can be used as a second probiotic composition. In some embodiments, the second probiotic composition comprises a bacterium from a genus Lactobacillus, e.g., L. paracasei, L. fermentum, L. acidophilus or L. reuteri. In other embodiments, the second probiotic composition comprises a bacterium from a genus Bifidobacterium, e.g., B. animalis, B. breve, B. lactis, B. longum. In some embodiments, the second probiotic composition comprises Lactobacillus paracasei and/or Lactobacillus fermentum.

Other ingredients (such as vitamin C, for example), may be included as oxygen scavengers and prebiotic substrates (such as these improve the colonization and survival in vivo).

### Antimicrobial Agents

In some embodiments, the methods described herein include administering one or more antimicrobial agents to treat a concurrent asthma infection, if present. The term “antimicrobial” refers to the ability of compounds to prevent, inhibit, delay or destroy the growth of microbes such as bacteria, fungi, protozoa and viruses.

In other embodiments, the agent is an antibacterial agent or an antibiotic agent. The term “antibacterial” or “antibiotic”, used herein interchangeably, refers to the ability of compounds to prevent, inhibit or destroy the growth of microbes of bacteria.

Any antibiotic or antibacterial agent effective against pathogenic bacteria, e.g., microorganisms of genus Moraxella, microorganisms genus Staphylococcus, microorganisms of genus Streptococcus, or other microorganisms present in the upper-airway of a subject being treated, may be used.

Non-limiting examples of antibiotic or antibacterial agents include Penicillin antibiotics such as, but not limited to, penicillin, methicillin, amoxicillin, ampicillin, flucloxacillin, penicillin C, penicillin V, carbenicillin, piperacillin, ticarcillin, oxacillin, dicloxacillin, azlocillin, cloxacillin, mezlocillin, temocillin, and nafcillin. Penicillin antibiotics may be used in combination with beta-lactamase inhibitors to provide broader spectrum activity, these combination antibiotics include amoxicillin/clavulanate, ampicillin/sulbactam, piperacillin/tazobactam, and clavulanate/ticarcillin.

Additional non-limiting examples of antibiotic or antibacterial agents include Cephalosporin antibiotics such as, but are not limited to, cefadroxil, cephradine, cefazolin, cephalexin, cefepime, ceftaroline, loracarbef, cefotetan, cefuroxime, cefprozil, cefoxitin, cefaclor, ceftibuten, cetriaxone, cefotaxime, cefpodoxime, cefdinir, cefixime, cefditoren, ceftizoxime, cefoperazone, cefalotin, cefamanadole, ceftaroline fosamil, cetobiprole, and ceftazidime. Cephalosporin antibiotics may be used in combination with beta-lactamase inhibitors to provide broader spectrum activity these combination antibiotics include, but are not limited to, avibactam/ceftazidime and ceftolozane/tazobactam.

Additional non-limiting examples of antibiotic or antibacterial agents include Sulfonamide antibiotics such as, but are not limited to, sulfamethoxazole, sulfasalazine, mafenide, sulfacetamide, sulfadiazine, silver sufadiazine, sulfadimethoxine, sulfanilimide, sulfisoxazole, sulfonamidochrysoidine, and sulfisoxazole. Sulfonamide antibiotics may be used in combination with trimethoprim to improve bactericidal activity.

Additional non-limiting examples of antibiotic or antibacterial agents include tetracycline antibiotics such as, but are not limited to, tetracycline, doxycycline, demeclocycline, minocycline, and oxytetracycline; Quinolone antibiotics such as, but are not limited to, lomefloxacin, ofloxacin, norfloxacin, gatifloxacin, ciprofloxacin, moxifloxacin, levofloxacin, gemifloxacin, cinoxacin, nalidixic acid, trovaloxacin, enoxacin, grepafloxacin, temafloxacin, and sparfloxacin; Oxaxolidinone antibiotics such as, but are not limited to, linezolid, posizolid, radezolid, and torezolid, Lincomycin antibiotics such as, but are not limited to, clindamycin and lincomycin; Macrolide antibiotics such as, but are not limited to, azithromycin, clarithromycin, erythromycin, telithromycin, dirithromycin, roxithromycin, troleandomycin, spiramycin, or fidazomycin; Glycopeptide antibiotics such as, but not limited to, dalbavancin, oritavancin, telavancin, teicoplanin, and vancomycin; Aminoglycoside antibiotics such as, but are not limited to, paromomycin, tobramycin, gentamicin, amikacin, kanamycin, neomycin, netilmicin, streptomycin and spectinomycin; Carbapenem antibiotics such as, but are not limited to, imipenem, meropenem, doripenem, ertapenem, and imipenem/cilastatin; Ansamycin antibiotics such as, but are not limited to, geldanamycin, herbimycin, and rifaximin; Lipopeptide antibiotics include, but are not limited to, daptomycin; Monobactam antibiotics such as, but are not limited to, aztreonam; Nitrofuran antibiotics such as, but are not limited to furazolidone and nitrofurantoin; and Polypeptide antibiotics such as, but are not limited to, bacitracin, colistin, and polymyxin B.

Other antibiotics which may be used include, but are not limited to, clofazimine, dapsone, capreomycin, cycloserine, ethambutol, ethionamide, isoniazid, pyrazinamide, rifampicin, rifabutin, rifapentine, streptomycin, arsphenamide, chloramphenicol, fosfomycin, fusidic acid, metronidazole, mupirocin, platensimycin, quinupristin/dalfopristin, thiamphenicol, tigecycline, tinidazole, and trimethoprim

### Pharmaceutical Compositions

The probiotic, prebiotic or antibiotic agents or formulations described herein can be administered alone or in a pharmaceutical composition comprising the probiotic, prebiotic or antibiotic agent as described herein, and one or more pharmaceutically acceptable excipients.

As used herein, the terms “composition” generally refers to any product comprising more than one ingredient. The compositions of the present disclosure are in biologically compatible form suitable for administration in vivo to subjects. The pharmaceutical compositions further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle.

In some embodiments, the formulations of the present disclosure are suitable for administration to an airway of a subject. In some embodiments, the agent is administered to an upper airway of the individual, e.g., the pharynx (e.g., nasopharynx, oropharynx, and laryngopharynx), the nasal cavity, the larynx or the trachea.

In some embodiments, the formulations of the present disclosure are in a unit dosage form suitable for inhalation. For inhalation formulations, the formulations provided herein may be delivered via any inhalation methods known to a person skilled in the art. Such inhalation methods and devices include, but are not limited to, metered dose inhalers with propellants such as chlorofluorocarbons (CFC) or hydrofluoroalkane (HFA) or other propellants that are physiologically and environmentally acceptable. Other suitable devices are breath operated inhalers, multidose dry powder inhalers and aerosol nebulizers. Aerosol formulations for use in the methods of the disclosure may include propellants, surfactants and co-solvents and may be filled into conventional aerosol containers that are closed by a suitable metering valve.

Inhalant compositions may comprise liquid or powdered compositions containing the active ingredient (e.g., a microorganism) that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions may be administered by means of a pump or squeeze-actuated nebulized spray dispenser, or by any other conventional means for causing or enabling the requisite dosage amount of the liquid composition to be inhaled. Suitable formulations, wherein the carrier is a liquid, for administration, as for example, a nasal spray or as nasal drops, include aqueous or oily solutions of the active ingredient. Alternatively, the composition may be a dry powder and administered to the respiratory tract.

In other aspects, the probiotic or prebiotic composition is formulated for oral administration. In one aspect, the probiotic or prebiotic composition is an orally administrable composition of metabolically active, i.e., live and/or or lyophilized, or non-viable heat-killed, irradiated or lysed probiotic bacteria. The probiotic or prebiotic composition may contain other ingredients.

For oral therapeutic administration, the active ingredient (e.g., microorganism) may be combined with one or more excipients and used in the form of ingestible tablets, buccal tablets, troches, capsules, powders, elixirs, suspensions, syrups, wafers, and the like. The percentage of the compositions and preparations may vary and may be between about 1 to about 99% weight of the active ingredient(s). Non-limiting examples of pharmaceutically-acceptable excipients suitable for use in the invention include binding agents, disintegrating agents, lubricants, sweetening agents, anti-adherents, anti-static agents, surfactants, anti-oxidants, coating agents, coloring agents, plasticizers, preservatives, suspending agents, emulsifying agents, spheronization agents, and any combination thereof.

The amount of active ingredient (e.g., microorganism) in such therapeutically useful compositions is such that an effective dosage level will be obtained. Suitable pharmaceutical excipients include cellulose, starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain wetting or emulsifying agents, or pH buffering agents. Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

As used herein, the term “administering” generally refers to any and all means of introducing compounds described herein to the host subject including, but not limited to, by administration by inhalation or oral administration. Microorganisms described herein may be administered in unit dosage forms and/or compositions containing one or more pharmaceutically-acceptable carriers, adjuvants, diluents, excipients, and/or vehicles, and combinations thereof.

Pharmaceutical compositions described herein can be in unit dosage forms suitable for single administration of precise dosages. In unit dosage form, the formulation is divided into unit doses containing appropriate quantities of one or more microorganisms. The unit dosage can be in the form of a package containing discrete quantities of the formulation.

### Dosing and Dosing Schedules

It will be understood, that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient), and the severity of the particular disorder undergoing therapy.

As used herein, the term “therapeutically effective dose” means (unless specifically stated otherwise) a quantity of a compound which, when administered either one time or over the course of a treatment cycle affects the health, wellbeing or mortality of a subject (e.g., and without limitation, delays the onset of and/or reduces the severity of one or more of the symptoms associated with asthma). Useful dosages of the microorganisms of the present disclosure can be determined by comparing their in vitro activity, and the in vivo activity in animal models. Methods of the extrapolation of effective dosages in mice and other animals to human subjects are known in the art. The amount of the composition required for use in treatment (e.g., the therapeutically or diagnostically effective amount or dose) will vary not only with the particular application, and the characteristics of the subject (such as, for example, age, condition, sex, the subject's body surface area and/or mass, tolerance to drugs) and will ultimately be at the discretion of the attendant physician, clinician, or otherwise.

A suitable dose of the probiotic bacteria is from about 1×10³ to about 1×10¹¹ colony forming units (CFU). In some embodiments, a suitable dose of the probiotic bacteria is from about 1×10⁴ to about 1×10¹¹ CFU. In some embodiments, a suitable dose of the probiotic bacteria is from about 1×10⁵ to about 1×10¹¹ CFU. In some embodiments, a suitable dose of the probiotic bacteria is from about 1×10⁶ to about 1×10¹¹ CFU. In some embodiments, a suitable dose of the probiotic bacteria is from about 1×10⁷ to about 1×10¹¹ CFU. In some embodiments, a suitable dose of the probiotic bacteria is from about 1×10⁸ to about 1×10¹¹ CFU. In some embodiments, a suitable dose of the probiotic bacteria is from about 1×10⁹ to about 1×10¹¹ CFU. In some embodiments, a suitable dose of the probiotic bacteria is from about 1×10¹⁰ to about 1×10¹¹ CFU. In some embodiments, a suitable dose of the probiotic bacteria is from about 1×10⁵ to about 1×10¹⁰ CFU. In some embodiments, a suitable dose of the probiotic bacteria is from about 1×10⁵ to about 1×10⁹ CFU. In some embodiments, a suitable dose of the probiotic bacteria is from about 1×10⁵ to about 1×10⁸ CFU. In some embodiments, a suitable dose of the probiotic bacteria is from about 1×10⁵ to about 1×10⁷ CFU. In some embodiments, a suitable dose of the probiotic bacteria is from about 1×10⁵ to about 1×10⁶ CFU.

A suitable daily dose of the probiotic bacteria is from about 1×10³ to about 1×10¹¹ colony forming units (CFU)/Kg, based on the weight of the formulation. In some embodiments, a suitable daily dose of the probiotic bacteria is from about 1×10⁴ to about 1×10¹¹ CFU/Kg, based on the weight of the formulation. In some embodiments, a suitable daily dose of the probiotic bacteria is from about 1×10⁵ to about 1×10¹¹ CFU/Kg, based on the weight of the formulation. In some embodiments, a suitable daily dose of the probiotic bacteria is from about 1×10⁶ to about 1×10¹¹ CFU/Kg, based on the weight of the formulation. In some embodiments, a suitable daily dose of the probiotic bacteria is from about 1×10⁷ to about 1×10¹¹ CFU/Kg, based on the weight of the formulation. In some embodiments, a suitable daily dose of the probiotic bacteria is from about 1×10⁸ to about 1×10¹¹ CFU/Kg, based on the weight of the formulation. In some embodiments, a suitable daily dose of the probiotic bacteria is from about 1×10⁹ to about 1×10¹¹ CFU/Kg, based on the weight of the formulation. In some embodiments, a suitable daily dose of the probiotic bacteria is from about 1×10¹⁰ to about 1×10¹¹ CFU/Kg, based on the weight of the formulation. In some embodiments, a suitable daily dose of the probiotic bacteria is from about 1×10⁵ to about 1×10¹⁰ CFU/Kg, based on the weight of the formulation. In some embodiments, a suitable daily dose of the probiotic bacteria is from about 1×10⁵ to about 1×10⁹ CFU/Kg, based on the weight of the formulation. In some embodiments, a suitable daily dose of the probiotic bacteria is from about 1×10⁵ to about 1×10⁸ CFU/Kg, based on the weight of the formulation. In some embodiments, a suitable daily dose of the probiotic bacteria is from about 1×10⁵ to about 1×10⁷ CFU/Kg, based on the weight of the formulation. In some embodiments, a suitable daily dose of the probiotic bacteria is from about 1×10⁵ to about 1×10⁶ CFU/Kg, based on the weight of the formulation.

In some embodiments, a suitable daily dose of the probiotic bacteria is about 0.01 mg, about 0.02 mg, about 0.03 mg, about 0.04 mg, about 0.05 mg, about 0.06 mg, about 0.07 mg, about 0.08 mg, about 0.09 mg about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 0.5 mg, about 10 mg, about 10.5 mg, about 11 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg.

In some embodiments, a suitable daily dose of the probiotic bacteria is from about 0.1 mg to about 100 mg; 0.1 mg to about 75 mg; from about 0.1 mg to about 50 mg; from about 0.1 mg to about 25 mg; from about 0.1 mg to about 10 mg; 0.1 mg to about 7.5 mg, 0.1 mg to about 5 mg; 0.1 mg to about 2.5 mg; from about 0.1 mg to about 1 mg; from about 0.5 mg to about 100 mg; from about 0.5 mg to about 75 mg; from about 0.5 mg to about 50 mg; from about 0.5 mg to about 25 mg; from about 0.5 mg to about 10 mg; from about 0.5 mg to about 5 mg, from about 0.5 mg to about 2.5 mg; from about 0.5 mg to about 1 mg; from about 1 mg to about 100 mg; from about 1 mg to about 75 mg; from about 0.1 mg to about 50 mg; from about 0.1 mg to about 25 mg; from about 0.1 mg to about 10 mg; from about 0.1 mg to about 5 mg; from about 0.1 mg to about 2.5 mg; from about 0.1 mg to about 1 mg.

In some embodiments, a suitable daily dose of a prebiotic agent, an antibiotic agent or a drug is about 0.01 mg, about 0.02 mg, about 0.03 mg, about 0.04 mg, about 0.05 mg, about 0.06 mg, about 0.07 mg, about 0.08 mg, about 0.09 mg about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 0.5 mg, about 10 mg, about 10.5 mg, about 11 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg.

In some embodiments, a suitable daily dose of a prebiotic agent, an antibiotic agent or a drug is from about 0.1 mg to about 100 mg; 0.1 mg to about 75 mg; from about 0.1 mg to about 50 mg; from about 0.1 mg to about 25 mg; from about 0.1 mg to about 10 mg; 0.1 mg to about 7.5 mg, 0.1 mg to about 5 mg; 0.1 mg to about 2.5 mg; from about 0.1 mg to about 1 mg; from about 0.5 mg to about 100 mg; from about 0.5 mg to about 75 mg; from about 0.5 mg to about 50 mg; from about 0.5 mg to about 25 mg; from about 0.5 mg to about 10 mg; from about 0.5 mg to about 5 mg, from about 0.5 mg to about 2.5 mg; from about 0.5 mg to about 1 mg; from about 1 mg to about 100 mg; from about 1 mg to about 75 mg; from about 0.1 mg to about 50 mg; from about 0.1 mg to about 25 mg; from about 0.1 mg to about 10 mg; from about 0.1 mg to about 5 mg; from about 0.1 mg to about 2.5 mg; from about 0.1 mg to about 1 mg.

The composition described herein can be administered by any dosing schedule or dosing regimen as applicable to the patient and/or the condition being treated. In some embodiments, the probiotic formulation is administered at least once a day. In other embodiments, the probiotic formulation is administered at least twice a day. In other embodiments, the probiotic formulation is administered at least thrice a day. In other embodiments, the probiotic formulation is administered once. In other embodiments, the probiotic formulation is administered once per week. In other embodiments, the probiotic formulation is administered at least twice per week. In other embodiments, the probiotic formulation is administered once per month. In other embodiments, the probiotic formulation is administered at least twice per month. In other embodiments, the probiotic formulation is administered on an as-needed basis to relieve symptoms of asthma.

### Additional Embodiments

In some aspects, provided herein is a method comprising (a) acquiring a biological sample from an upper airway of an individual with asthma, who are asymptomatic or symptomatic, (b) identifying a plurality of microorganisms in the biological sample, and (c) determining an amount, concentration, or proportion of a microorganism in the biological sample, wherein the microorganism in the biological sample is selected from a Moraxella species M. catarrhalis, M. nonliquefaciens, or M. lincolnii, a Staphylococcus species S. aureus or S. epidermidis, a Streptococcus species S. pneumoniae, S. sanguinis, S. mitis, S. infantis or S. oralis, a Corynebacterium species C. propinquum, C. pseudodiphtheriticum, or C. accolens, and a Dolosigranulum species D. pigrum.

In some aspects, provided herein is a method comprising (a) acquiring a biological sample from an upper airway of an individual with early signs of loss of asthma control, (b) identifying a plurality of microorganisms in the biological sample, and (c) determining an amount, concentration, or proportion of a microorganism in the biological sample, wherein the microorganism in the biological sample is selected from a Moraxella species M. catarrhalis, M. nonliquefaciens, or M. lincolnii, a Staphylococcus species S. aureus or S. epidermidis, a Streptococcus species S. pneumoniae, S. sanguinis, S. mitis, S. infantis or S. oralis, a Corynebacterium species C. propinquum, C. pseudodiphtheriticum, or C. accolens, and a Dolosigranulum species D. pigrum.

In some embodiments, an increased amount, concentration, or proportion of a microorganism of a Moraxella species M. catarrhalis, M. nonliquefaciens, or M. lincolnii, a Staphylococcus species S. aureus or S. epidermidis or a Streptococcus species S. pneumoniae, S. sanguinis, S. mitis, S. infantis or S. oralis, or a decreased amount, concentration, or proportion of a Corynebacterium species C. propinquum, C. pseudodiphtheriticum, or C. accolens, or a Dolosigranulum species D. pigrum is indicative of an increased risk of asthma exacerbations, a decreased time to loss of asthma control, or an increased risk of loss of asthma control in the subject.

In other aspects, provided herein is a method of treating an individual with early signs of loss of asthma control comprising (a) detecting an altered amount, concentration, or proportion of a microorganism in a biological sample from an upper airway of the individual; and (b) administering to the individual an agent that is effective to prevent or attenuate asthma exacerbations. In some embodiments, the microorganism is selected from the group consisting of microorganisms of genus Moraxella, microorganisms of genus Staphylococcus, microorganisms of genus Streptococcus, microorganisms of genus Corynebacterium, and microorganisms of genus Dolosigranulum. In some embodiments, the microorganism in the biological sample is selected from a Moraxella species M. catarrhalis, M. nonliquefaciens, or M. lincolnii, a Staphylococcus species S. aureus or S. epidermidis, a Streptococcus species S. pneumoniae, S. sanguinis, S. mitis, S. infantis or S. oralis, a Corynebacterium species C. propinquum, C. pseudodiphtheriticum, or C. accolens, and a Dolosigranulum species D. pigrum. In some embodiments, the microorganism comprises comprise 16S RNA sequences with 99% identity to any one of SEQ. ID No. 7-9.

In other aspects, provided herein is a method of treating an individual with asthma comprising (a) detecting an increased amount, concentration, or proportion of a microorganism of genus Moraxella, genus Staphylococcus or genus Streptococcus compared to an amount, concentration, or proportion of a microorganism of genus Corynebacterium or genus Dolosigranulum in a biological sample from an upper airway of the individual; and (b) administering to the individual an agent that is effective to prevent or attenuate asthma exacerbations. In some embodiments, the microorganism is selected from the group consisting of microorganisms of genus Moraxella, microorganisms of genus Staphylococcus, microorganisms of genus Streptococcus, microorganisms of genus Corynebacterium, and microorganisms of genus Dolosigranulum. In some embodiments, the microorganism in the biological sample is selected from a Moraxella species M. catarrhalis, M. nonliquefaciens, or M. lincolnii, a Staphylococcus species S. aureus or S. epidermidis, a Streptococcus species S. pneumoniae, S. sanguinis, S. mitis, S. infantis or S. oralis, a Corynebacterium species Corynebacterium species C. propinquum, C. pseudodiphtheriticum, or C. accolens, and a Dolosigranulum species D. pigrum.

In some embodiments, the agent is a prebiotic composition. In other embodiments, the agent is a probiotic composition. In some embodiments, the probiotic composition comprises an isolated microorganism of genus Corynebacterium, genus Dolosigranulum, or a combination thereof. In some embodiments, the isolated microorganism is a purified microorganism. In other embodiments, the microorganism is a Corynebacterium species Corynebacterium species C. propinquum, C. pseudodiphtheriticum, or C. accolens. In other embodiments, the microorganism is a Dolosigranulum species D. pigrum. In some embodiments, the microorganism comprises comprise 16S RNA sequences with 99% identity to any one of SEQ. ID. NOs. 7-9.

In other aspects, provided herein is a method of modifying a nasal microbiota of an individual comprising administering to the individual a probiotic composition comprising an isolated microorganism at a dose sufficient to cause an increase in an abundance or proportion of the microorganism in the nasal microbiota of the individual, wherein the isolated microorganism is a microorganism of genus Corynebacterium or a microorganism of genus Dolosigranulum.

In other aspects, provided herein is a probiotic composition for use in a method of modifying a nasal microbiota of an individual, the probiotic composition comprising an isolated microorganism at a dose sufficient to cause an increase in an abundance or proportion of the microorganism in the nasal microbiota of the individual, wherein the isolated microorganism is a microorganism of genus Corynebacterium or a microorganism of genus Dolosigranulum.

In some embodiments, the nasal microbiota of the individual comprises an elevated abundance or proportion relative to a reference sample of one or more microorganisms of genus Moraxella, genus Staphylococcus or genus Streptococcus, or a reduced abundance or proportion relative to the reference sample of one or more microorganisms of genus Corynebacterium or genus Dolosigranulum. In some embodiments, the microorganism is a Moraxella species M. catarrhalis, M. nonliquefaciens, or M. lincolnii, a Staphylococcus species S. aureus or S. epidermidis, a Streptococcus species S. pneumoniae, S. sanguinis, S. mitis, S. infantis or S. oralis, a Corynebacterium species Corynebacterium species C. propinquum, C. pseudodiphtheriticum, or C. accolens, and a Dolosigranulum species D. pigrum. The reference sample may be obtained from an individual not diagnosed with asthma.

In other aspects, provided herein is a method of treating an individual with a nasal microbiome characterized by an elevated abundance or proportion relative to a reference sample of one or more microorganisms of genus Moraxella, genus Staphylococcus or genus Streptococcus; or a reduced abundance or proportion relative to the reference sample of one or more microorganisms of genus Corynebacterium or genus Dolosigranulum, the method comprising administering to the individual a probiotic composition comprising an isolated microorganism, wherein the isolated microorganism is a microorganism of genus Corynebacterium or genus Dolosigranulum, thereby preventing, delaying or attenuating an asthma exacerbation of the individual. In some embodiments, the microorganism is selected from a Moraxella species M. catarrhalis, M. nonliquefaciens, or M. lincolnii, a Staphylococcus species S. aureus or S. epidermidis, a Streptococcus species S. pneumoniae, S. sanguinis, S. mitis, S. infantis or S. oralis, a Corynebacterium species Corynebacterium species C. propinquum, C. pseudodiphtheriticum, or C. accolens, and a Dolosigranulum species D. pigrum. The reference sample may be obtained from an individual not diagnosed with asthma.

In other aspects, provided herein is probiotic composition for use in a method of treating an individual with a nasal microbiome characterized by an elevated abundance or proportion relative to a reference sample of one or more microorganisms of genus Moraxella, genus Staphylococcus or genus Streptococcus; or a reduced abundance or proportion relative to the reference sample of one or more microorganisms of genus Corynebacterium or genus Dolosigranulum, the probiotic composition comprising an isolated microorganism, wherein the isolated microorganism is a microorganism of genus Corynebacterium or genus Dolosigranulum. In some embodiments, the microorganism is selected from a Moraxella species M. catarrhalis, M. nonliquefaciens or M. lincolnii, a Staphylococcus species S. aureus or S. epidermidis, a Streptococcus species S. pneumoniae, S. sanguinis, S. mitis, S. infantis or S. oralis, a Corynebacterium species Corynebacterium species C. propinquum, C. pseudodiphtheriticum, or C. accolens, and a Dolosigranulum species D. pigrum. The reference sample may be obtained from an individual not diagnosed with asthma.

In other aspects, provided herein is a method of treating an individual with a nasal microbiome characterized by an elevated abundance or proportion relative to a reference sample of one or more microorganisms of genus Moraxella, genus Staphylococcus or genus Streptococcus; or a reduced abundance or proportion relative to the reference sample of one or more microorganisms of genus Corynebacterium or genus Dolosigranulum, the method comprising administering to the individual a probiotic composition comprising an isolated microorganism, wherein the isolated microorganism is a microorganism of genus Corynebacterium or genus Dolosigranulum, thereby preventing or treating an upper respiratory tract infection of the individual. In some embodiments, the microorganism in the biological sample is selected from a Moraxella species M. catarrhalis, M. nonliquefaciens or M. lincolnii, a Staphylococcus species S. aureus or S. epidermidis, a Streptococcus species S. pneumoniae, S. sanguinis, S. mitis, S. infantis or S. oralis, a Corynebacterium species Corynebacterium species C. propinquum, C. pseudodiphtheriticum, or C. accolens, and a Dolosigranulum species D. pigrum. The reference sample may be obtained from an individual not diagnosed with asthma.

In other aspects, provided herein is probiotic composition for use in a method of treating an individual with a nasal microbiome characterized by an elevated abundance or proportion relative to a reference sample of one or more microorganisms of genus Moraxella, genus Staphylococcus or genus Streptococcus; or a reduced abundance or proportion relative to the reference sample of one or more microorganisms of genus Corynebacterium or genus Dolosigranulum, the probiotic composition comprising an isolated microorganism, wherein the isolated microorganism is a microorganism of genus Corynebacterium or genus Dolosigranulum, thereby preventing or treating an upper respiratory tract infection of the individual. In some embodiments, the microorganism in the biological sample is selected from a Moraxella species M. catarrhalis, M. nonliquefaciens, or M. lincolnii, a Staphylococcus species S. aureus or S. epidermidis, a Streptococcus species S. pneumoniae, S. sanguinis, S. mitis, S. infantis or S. oralis, a Corynebacterium species C. propinquum, C. pseudodiphtheriticum, or C. accolens, and a Dolosigranulum species D. pigrum. The reference sample may be obtained from an individual not diagnosed with asthma.

In some aspects, the present disclosure relates to a bacterial extract for administration to an upper airway of an individual with asthma, wherein the bacterial extract is (a) isolated from a microorganism of genus Corynebacterium or genus Dolosigranulum, and (b) capable of inhibiting growth and/or colonization of the upper airway of the individual by a microorganism of genus Staphylococcus, genus Streptococcus, or genus Moraxella. In other embodiments, the bacterial extract is secreted by the microorganism of Corynebacterium species C. propinquum, C. pseudodiphtheriticum, or C. accolens. In other embodiments, the bacterial extract is secreted by the microorganism of Dolosigranulum species D. pigrum. In some embodiments, the microorganism comprises comprise 16S RNA sequences with 99% identity to any one of SEQ. ID. NOs. 7-9.

In some aspects, provided herein is a method of treating an individual comprising administering the bacterial extract of the present disclosure an upper airway of the individual, thereby reducing an amount or proportion of a microorganism of genus Staphylococcus, genus Streptococcus, or genus Moraxella in an upper respiratory tract of the individual. In some embodiments, the microorganism is selected from a Moraxella species M. catarrhalis, M. nonliquefaciens, or M. lincolnii, a Staphylococcus species S. aureus or S. epidermidis, and a Streptococcus species S. pneumoniae, S. sanguinis, S. mitis, S. infantis or S. oralis.

Thus, in some aspects, provided herein is a probiotic formulation for preventing, delaying or attenuating asthma exacerbations comprising a pharmaceutically acceptable unit dose of a probiotic composition comprising one or more isolated microorganisms selected from microorganisms of genus Corynebacterium or genus Dolosigranulum, and a pharmaceutically acceptable carrier. In some embodiments, the bacterial extract is secreted by the microorganism of Corynebacterium species C. propinquum, C. pseudodiphtheriticum, or C. accolens. In other embodiments, the bacterial extract is secreted by the microorganism of Dolosigranulum species D. pigrum.

### EXAMPLES

### Example 1: Characterization of Nasal Microbiome of Individuals with Asthma

### Abbreviations:

### CI: Confidence Interval

### FEV1: Forced Expiratory Volume

### FVC: Forced Vital Capacity

### ICS: Inhaled Corticosteroid

### OCS: Oral Corticosteroids

### OR: Odds Ratio

### YZ: Yellow Zone

A cohort of school-age children with mild-moderate persistent asthma treated with daily ICS in a clinical trial was utilized to investigate whether the upper-airway microbiota at the time of respiratory health (randomization) is related to the development of future Yellow Zone (YZ) episodes, and whether the microbiome at YZ are related to the likelihood of progression to severe asthma exacerbation requiring oral corticosteroids (OCS).

### Characterization of the Airway Microbiota at Randomization

To identify any cohort characteristics that affects airway microbiota, Permutational multivariate ANOVA (PERMANOVA) was performed using nasal blow samples collected at time of randomization (RD) from 214 children participating in a clinical trial (mean age 8.0+/−1.8 years, 50% were males, and 57% were Caucasian). The 214 children are a representative of the total clinical trial cohort (n=254) as demographics and clinical characteristics were not statistically different between the 254 children who participated in the clinical trial and this subset of 214 children.

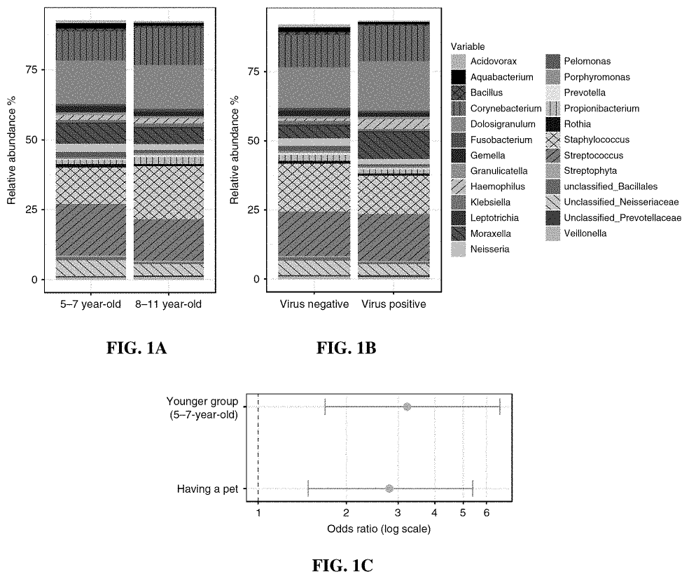

The overall microbial composition and abundances were significantly different in age groups (p=0.02, PERMANOVA). Differential abundance analysis by DESeq showed that older children (8-11-year-old) had higher randomization abundance of Staphylococcus (p=0.04), while younger children (5-7-year-old) had higher abundances of Moraxella (p=0.05) and Streptococcus (p=0.03) (FIG. 1A). Younger participants (5-7-year-old) were more likely to develop YZ (OR=3.2, 95% CI 1.7-6.8, p=0.003) compared to older participants. The presence of a pet in the house was associated with an increased risk of developing YZ (OR=2.8, 95% CI 1.5-5.4, p=0.006; (FIG. 1B)). 22.9% of the participants were treated with antibiotics during the 6 months prior to randomization, but this covariate did not affect the composition of the microbiota at time of randomization (p=0.52, PERMANOVA). Other than age or the presence of pets, no other clinical variables (gender, ethnicity, BMI, total IgE level, number of asthma-related hospitalizations in the past year, peripheral eosinophil count, and lung function values (FEV1 percent, FEV1/FVC ratio) were found to be associated with risk of developing YZ. Based on these findings, all subsequent analyses that included randomization samples were adjusted for age and the presence of pets at home.

Viral respiratory infections contribute to asthma exacerbations. Thus, the effect of respiratory viruses was evaluated. Respiratory viruses were detected in 33% of the randomization samples: 64% of these viruses were rhinovirus. The proportion of virus positive samples was numerically higher in younger age group (39.3% in young vs. 28.0% in old groups), but was not significantly different (p=0.11, Chi-squared test). Compared to viral negative samples, the relative abundance of Moraxella (p=0.04, by DESeq) were higher while the relative abundances of Bacillus (p=0.03, by DESeq) and Staphylococcus (p=0.04, by DESeq) were significantly lower in the viral positive samples, suggesting a possible interaction between viruses and these bacterial genera (FIG. 1B). However, the presence of respiratory virus at randomization (while the children did not have respiratory symptoms) was not associated with the risk of future YZ development (OR=1.4, 95% CI 0.72-2.90, p=0.33), or the risk of future exacerbations (OR=1, 95% CI 0.39-2.44, p=0.9). The finding that respiratory viruses were not predictive at baseline for subsequent loss of control and exacerbations may in part be attributable to the lack of differentiation of rhinovirus types as in children Rhinovirus C has been shown to be most strongly associated with asthma exacerbations compared to Rhinovirus A or B10,11. Nevertheless, all subsequent analyses that included randomization samples were adjusted for the presence of respiratory viruses at randomization.

### Baseline Airway Microbiota and Future Loss of Asthma Control.

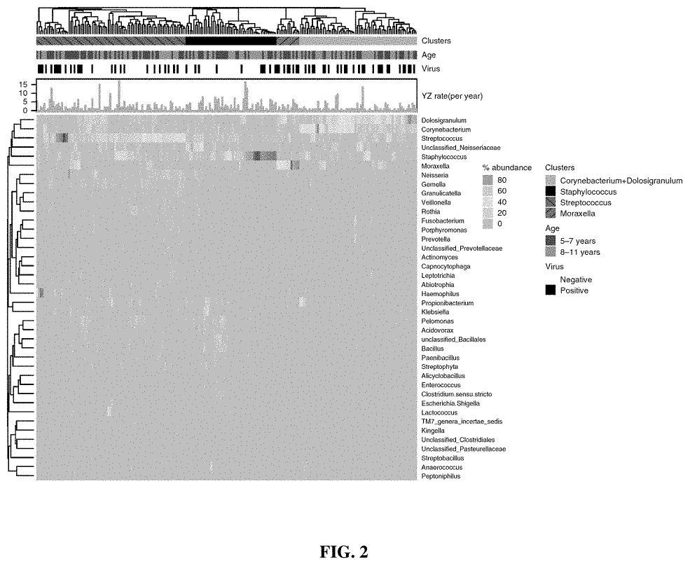

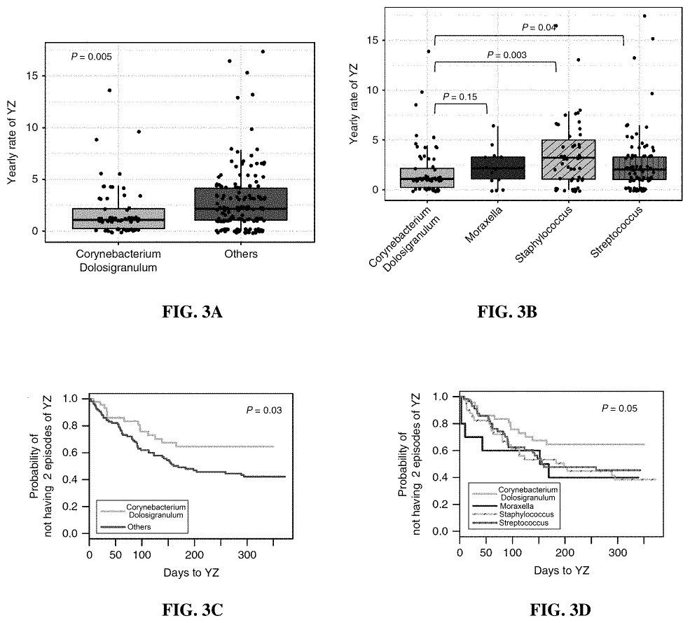

To evaluate whether the microbiota at time of well-controlled asthma (randomization) is related to loss of asthma control at YZ, unsupervised hierarchical clustering analysis was performed using 214 nasal samples from randomization. Four clusters were identified which were dominated by the following genera: Corynebacterium+Dolosigranulum, Staphylococcus, Streptococcus, and Moraxella (FIG. 2). During the follow up period (320 days), 75.7% and 43.4% of the participants experienced at least one and two episodes of YZ, respectively. The median annualized rate of YZ was significantly lower in participants who had the Corynebacterium+Dolosigranulum dominated cluster at randomization compared to the aggregate of the 3 other clusters combined together, or any other cluster (FIGS. 3A and 3B; p=0.005; Wilcoxon rank-sum test). In addition, Cox Proportional-Hazards analysis showed that participants in the Corynebacterium+Dolosigranulum cluster had significantly longer time to develop at least 2 episodes of YZ compared to participants in the combined 3 other clusters (FIG. 3C, p=0.03, ward test) and compared to any other cluster (FIG. 3D, p=0.05, ward test). Time to the first episode of YZ, was not statistically different between the groups.

Changes in Airway Microbiota from Randomization to YZ.

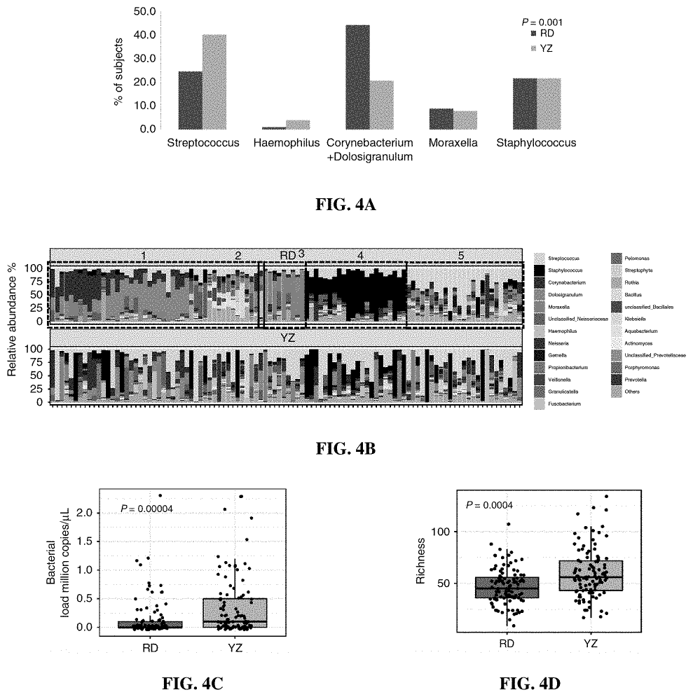

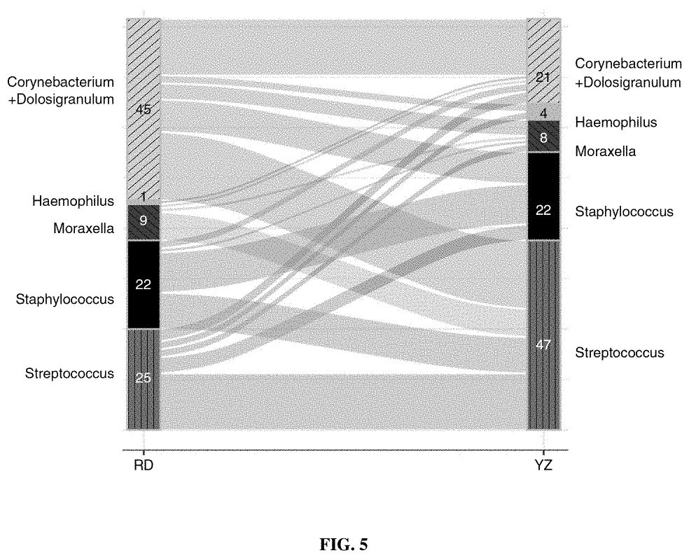

The dynamics of nasal bacterial microbiota from randomization to YZ was determined utilizing samples from the 102 participants who contributed samples both at randomizing and at YZ (102 paired samples (total 204 samples)). The bacterial compositions and their relative abundance in individual patients demonstrated profound alteration from randomization to YZ (FIG. 4B, FIG. 5). Surprisingly, more than half of the patients switched to a different dominant cluster between these 2 time points, most commonly to the Streptococcus cluster (FIG. 5). Consequently, the Streptococcus cluster became the most prevalent cluster at YZ (FIG. 4A). The changes of microbiota are also evident in bacterial alpha diversity and total bacterial load. At YZ, total bacterial load (FIG. 4C) and bacterial richness (FIG. 4D) were significantly higher (p<0.01 for both, Wilcoxon rank-sum test) than those at randomization.

### Airway Microbiota at YZ and Asthma Exacerbations.

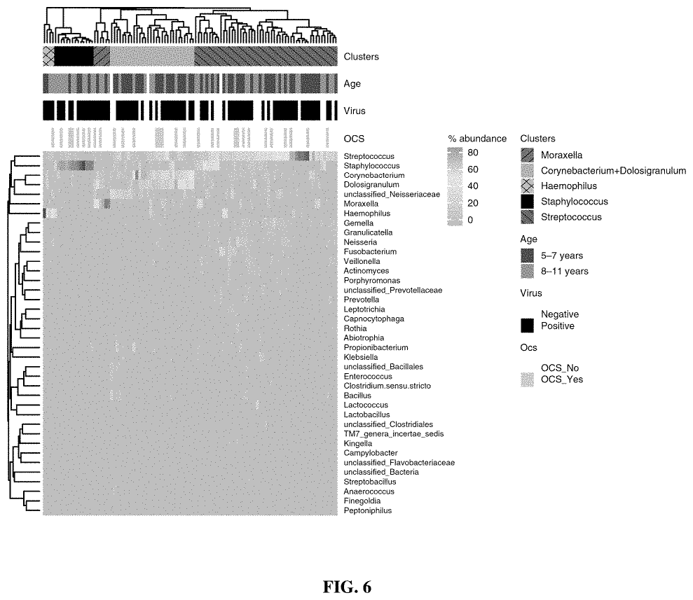

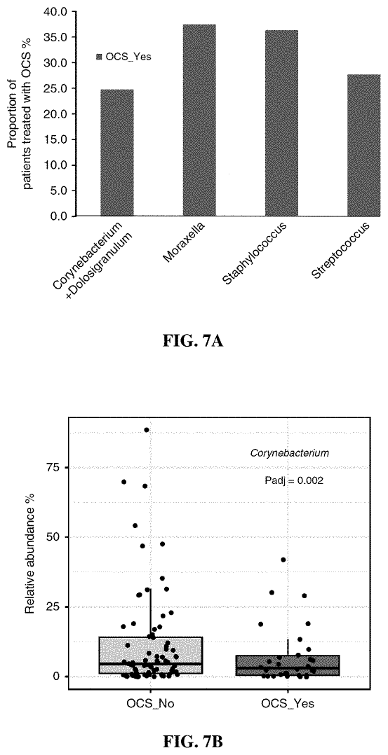

To test whether the microbiome at the time of YZ is associated with severe exacerbation, the microbiota in participants who progressed to severe exacerbation requiring OCS therapy ( 30/105=28.6%) was compared with those who did not ( 75/105=71.4%) (FIG. 6). A numerically lower, but not statistically significant proportion of patients who required OCS from the Corynebacterium+Dolosigranulum cluster at the time of YZ was identified compared to the other three clusters (FIG. 7A, p=0.35, Chiseq-test). Furthermore, analysis of specific bacterial genera revealed that Corynebacterium was more abundant at YZ in samples obtained from episodes that did not progress to severe exacerbation (FIG. 7B, P=0.002, DeSeq). In addition, higher relative abundance of Corynebacterium was associated with a modest reduction in the risk of progressing to exacerbation (OR=0.92, 95% CI 0.89-0.94, P=0.04). No association was identified between that microbiome at randomization and severe exacerbations, likely due to the profound changes in the microbiome between RD and YZ. Finally, bacterial richness (p=0.64, Wilcoxon rank-sum test) or load (p=0.16, Wilcoxon rank—sum test) at Yellow Zone were not associated with asthma exacerbations.

### Dynamic Change of the Microbiota and Asthma Exacerbations.

Given the upper airway microbiota of most participants changed from randomization to YZ (FIG. 5), it was further revaluated whether the dynamic change of the microbiota, namely, switching to a different cluster or maintaining of the same cluster, was associated with exacerbation risk. Switching from Corynebacterium+Dolosigranulum cluster at randomization to the Moraxella cluster at YZ was associated with the highest risk of exacerbation compared to the other combinations of cluster changes (P=0.04, Chiseq-test).

### Respiratory Viruses at YZ and Asthma Exacerbations.

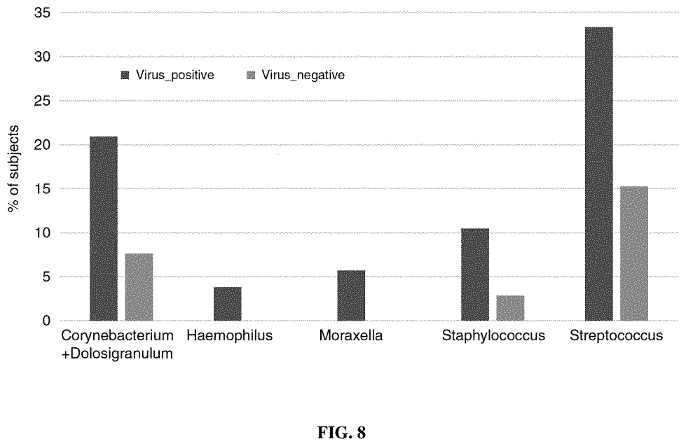

The potential contribution of respiratory viruses at time of YZ to asthma exacerbations was evaluated. Respiratory viruses were detected in 78 (74.3%) of the YZ samples, of these 48 samples were enterovirus/human rhinovirus (EV/RV) positive. Presence of virus was not associated with severe exacerbations. Samples that belonged to Moraxella (n=4) and Haemophilus clusters (n=6) were all virus positive (FIG. 8). Most of the samples assigned into Moraxella and Haemophilus dominated microbial clusters at YZ switched from a non-Moraxella and non-Haemophilus clusters at RD (FIG. 5), reflecting the emergence of Moraxella and Haemophilus communities at the time of YZ.

### Discussion

The present disclosure provides characterization of the upper-airway bacterial microbiota of school-age children receiving daily low-dose ICS at time of well-controlled asthma (randomization) and during loss of asthma control (YZ). As demonstrated herein, airway microbiota colonization patterns were differentially associated with risk of loss of asthma control and severe exacerbation. The airway microbiota of children classified as dominated by Corynebacterium+Dolosigranulum genera during randomization was associated with a lower risk of developing loss of asthma control compared to those with microbiota being dominated by more pathogenic bacteria, specifically Staphylococcus, Streptococcus, and Moraxella. Furthermore, at the time of YZ, Corynebacterium's relative abundance was inversely associated with the likelihood of progressing from YZ to severe exacerbation.

The findings provided in the present disclosure suggest the potential pathogenic role of Streptococcus, Moraxella, and Haemophilus in asthma. The results extend the potential pathogenic role of Moraxella to the large population of children with mild asthma, which are the vast majority of childhood asthma patients. Further, the data shows that Staphylococcus is associated with highest YZ episodes, which is different from previous studies. The vast majority of Staphylococcus genera were comprised of Staphylococcus aureus.

The present disclosure identifies an association between commensal bacteria such as Corynebacterium and Dolosigranulum and asthma control. Corynebacterium is the most abundant genus identified in the nose by the Human Microbiome Project that characterized the normal microbial composition in healthy adults, and was found less frequently as a dominant member of the nasal microbiome in asthmatic adults, suggesting that these bacteria may have a protective effect. Since bacteria colonizing the upper airways, as those colonizing other niches within the human body, exist in a competitive state, it is plausible that competitive colonization may be one of the factors by which commensal bacteria provide protection against pathogen colonization and overgrowth. Indeed, Corynebacterium and Dolosigranulum can inhibit the growth of Streptococcus by releasing antibacterial products that may prevent nasal colonization with Streptococcus. Collectively these observations indicate that from an ecological perspective, a microbiome at equilibrium may resist colonization with pathogenic bacteria and is important for the maintenance of a healthy airway. This hypothesis is supported by the findings of the present disclosure, as the composition of airway microbiota at time of respiratory health was associated with loss of asthma control during the following year. Thus, the relative abundances of microorganisms such as Corynebacterium and Dolosigranulum may be potential microbiome markers at respiratory health to predict loss of asthma control in the future.

The airway-microbiota composition dramatically changed between randomization and YZ visits. The directionality of switch was found to be important, since switching from the microbiota dominated by Corynebacterium/Dolosigranulum to Moraxella cluster was associated with higher risk of asthma exacerbation. This observation indicates that commensal nasal microbiota in asthmatic children does not appear to prevent overgrowth by all pathogenic bacteria, in particular Moraxella, suggesting distinct microbial interactions between specific members of the airway microbiota. In addition, respiratory viruses, mainly rhinovirus, were detected in most YZ samples. As demonstrated herein, samples belonging to the Moraxella or Haemophilus dominated microbiota during YZ were all positive for respiratory viruses. This finding is consistent with the high rate of virus-positive samples in children with these microbiota signatures at randomization, and with previous reports linking rhinovirus infection with the detection of these bacterial genera in the airway.

The present disclosure demonstrates that some bacteria are associated with favorable asthma outcomes, while other bacteria are associated with asthma morbidity. The advantages of this study are mainly related to its conduct as a study coupled to a well-designed clinical trial. Patients were carefully characterized resulting in a homogenous population of school-aged children all requiring step 2 asthma care, treated with an identical dose of ICS. Therefore, microbiota differences that may be related to disease severity and/or effects of different ICS dosing, which are factors known to affect the airway microbiome, were minimized. Prospective data and sample collection as part of a clinical trial together with tight follow-up visits and calls have minimized recall and measurement biases. Finally, YZ samples were collected before applying study intervention (high-dose ICS), eliminating the effect of high-dose ICS on the microbiome.

The present disclosure identified associations between bacterial clusters defined by relative abundance of bacteria and clinical outcomes. Determining an absolute density of different bacteria in a metagenomic sample is an emerging concept in microbiome research. Relative and absolute abundances of the microbiome may be complementary to each other. As a low biomass material, bacterial density from nasal wash warrants thorough investigation regarding methodology including efficiency and robustness and data normalization for microbiome analysis. In addition, functional level characterization of the bacterial microbiome by transcriptome analysis or IgA-Seq are likely to improve our understanding of the role of airway microbiome in asthma and its exacerbation.

This study revealed that changes in the upper respiratory tract are associated with events in the lower respiratory tract. Although there are similarities between the lung and upper-airway microbiota compositions, studies have shown that these different compartments have different microbiome composition. Nevertheless some key bacterial taxa co-exist in the nasal and bronchial airways, especially in asthmatics. In addition, multiple studies have highlighted the relevance of the upper airway microbiota as a surrogate for the lung microbiota, and have shown that the upper-airway is a relevant compartment that provides valuable data on asthma inception, asthma diagnosis, and asthma exacerbations.

In summary, the present disclosure demonstrates a relationship between upper-airway microbiota composition and the risk of both loss of asthma control and severe exacerbations, among school-aged children with asthma. A randomization upper airway microbiota dominated by Corynebacterium+Dolosigranulum was associated with a significantly lower rate of YZ development. In addition, upper-airway microbiota composition was not static, and a shift to a Moraxella-dominant microbiome at YZ and/or a lower Corynebacterium abundance at YZ were both associated with increased risk of severe exacerbations during the following year.

### Example 2—Methods

Study design and participants. This microbiome study was a microbiome study coupled to the Step Up Yellow Zone Inhaled Corticosteroids to Prevent Exacerbations (STICS; NCT02066129) clinical trial, conducted by the NHLBI's AsthmaNet. The STICS clinical trial investigated whether, in school age children with mild-moderate persistent asthma who are treated with daily low-dose ICS, quintupling the dose of ICS in the YZ would reduce the rate of severe asthma exacerbations treated with oral corticosteroids. In the study, 254 children, 5-11 years of age, were treated for 48 weeks with maintenance open-label low-dose inhaled glucocorticoids (fluticasone propionate, 88 μg twice daily) and were randomly assigned to receive either the same ICS dose or use a quintupled dose ICS for 7 days at the early signs of loss of asthma control (YZ). The primary outcome was the rate of severe asthma exacerbations treated with systemic glucocorticoids, which were prescribed as a rescue therapy based on pre specified protocol criteria. The rate of severe asthma exacerbations was not different between the groups.

Nasal blow samples for microbiome studies were obtained at 2 time points: (1) At the randomization visit (RD) once the child had no respiratory symptoms; and (2) at the time of the first episode of early signs of loss of asthma control (YZ) prior to starting the YZ intervention (regular or high-dose ICS). The second sample was obtained before starting the YZ intervention in order to avoid potential effect of high-dose ICS on the nasal microbiota. The YZ sample was obtained by the parents at home based on instructions received at the randomization visit. The nasal samples were analyzed for common respiratory viruses by multiplex polymerase chain reaction (PCR).

16S rRNA Gene Sequencing and Normalization.

Total genomic DNA was extracted from 200 μl nasal blow samples using the bioMerieux NucliSENS easy-MAG automated extractor kit following standard protocol. We followed standard Illumina sequencing protocol. To characterize the bacterial microbiota, the V1 to V3 regions of 16S rRNA gene were amplified (primers 27F and 534R (27F:5′-AGAGTTTGATCCTGGCTCAG-3′ (SEQ ID NO: 10) and 534R: 5′-ATTACCGCGGCTGC TGG-3′ (SEQ ID NO: 11)), barcoded, and sequenced on the Illumina Miseq (2×300 bp) platform. Paired-end reads were assembled using Flash V1.2.7. Assembled reads were assigned to taxonomies using Ribosomal Database Project (RDP) software with classification confidence at >=0.8. The processed reads were subsampled to 10,000 reads/sample for 16S rRNA gene sequences. As an extraction control, PCR negative control was used for DNA extraction and sequencing. Less than five hundred reads were found in these negative controls, suggesting background noise is less likely to have significant impact on the data analysis. To minimize the effect of background noise on data analysis, we taxa that are potentially contamination from downstream analysis were removed. These taxa include unclassified Bradyrhizobiaceae, Thermohydrogenium, Aquabacterium, unclassified burkholderiales, Brevundimonas, Rhizobium, and Soonwooa.

Taxa with >=0.1% relative abundance were used (after removing the contamination taxa discussed above) to construct the heatmaps at randomization and Yellow Zone. These taxa accounts for 99% of total bacterial abundance on average (the minimal coverage is 82%).

To classify Staphylococcus to species level, all the reads that mapped to Staphylococcus genus were blasted to 16S rRNA database in NCBI. V13 reads with ˜500 bp in length of Staphylococcus aligned to S. aureus with 100% coverage, 100% identity, which is distinct from S. epidermidis (97% identity).

### Quantification of Bacterial Load.

Quantification of the bacterial 16S rRNA gene copy number in nasal blow samples was performed using a modification of the BactQuant assay. Total nucleic acids were extracted from the samples using the automated BioMerieux NucliSens easyMAG extractor. The BactQuant quantitative PCR was performed on the extracted DNA as described by Liu et al. (BMC Micobiol. (2012), 12, 56) with the following modifications: total reaction volume was 20 μl, including 3 μl of extract, and the assay was performed on an Applied Biosystems 7500 Real TimePCR System instrument. Quantification standards consisted of dilutions of a plasmid containing the E. coli 16S rRNA gene with results being expressed as copies of 16S rRNA gene per microliter.

### Statistical Analysis of the Microbiome Data.

The processed reads were sub-sampled to 10,000 reads/sample for 16S rRNA gene sequences. The abundance of at axon in a sample was represented as the relative abundance, which was calculated by dividing the number of reads assigned to a taxon by the total read counts, divided by 10,000, of the sample.

Exploratory multivariable analysis and formal statistical testing were performed. Exploratory multivariable analysis was done through hierarchical clustering to identify microbiome distribution patterns. Taxa with the relative abundances >0.1% was included in hierarchical clustering. Complete linkage was used for assigning samples to clusters. R package Complex Heatmap was used for cluster visualization and annotation of clinical variables. The names of clusters are defined based on the dominant bacteria genus in for that cluster. Permutational multivariate ANOVA (PERMANOVA) was used for formal statistical testing to investigate whether the bacterial community structure varied between different clinical parameters. DESeq2 was used to identify differential taxa between samples at randomization (RD) and YZ. Clinical variables including age, viral infection, gender and having a pet were first tested individually using PERMANOVA or DESeq2. The confounding variables were included along with variable of interest (RD and YZ) in the final model of PERMANOVA or DESeq2. The results from DESeq2 were further speculated by plotting the raw and relative abundance data. Results that are likely driven by outliers were removed from final reporting. Bacterial diversity including Richness and Shannon Diversity was computed using R package Vegan and statistical significance between groups was determined using Wilcoxon-rank test. Comparison of patient numbers between clusters was performed by chi-square or Fisher's exact test. Exacerbation data was treated as categorical data (0 and 1) and was applied to a generalized logistic regression model with binomial distribution to determine whether a given taxon is associated with exacerbation outcome. Odd-Ratios (ORs) were evaluated using generalized logistic regression. Multivariate models of Cox Proportional-Hazards analysis were performed to assess the association between the microbiome clusters at randomization and the development of >=2 episodes of YZ after adjustment for age and the presence of pets. Kaplan-Meier survival analysis was also performed to view the results from Cox Proportional-Hazards analysis. A P value of less than 0.05 was considered statistically significant in all the analysis. P values were corrected by false discovery rate when multiple comparisons were involved. All the analyses, described above, were performed in R (version 3.2.2).

While certain embodiments of the present disclosure have been shown and described herein, it will be apparent to those skilled in the art that such embodiments are provided by way of example only. It is not intended that the invention be limited by the specific examples provided within the specification.

While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-116. (canceled)

117. A method comprising: administering to an individual a composition comprising, or a bacterial extract obtained from, Corynebacterium microorganisms and Dolosigranulum microorganisms at a dose sufficient to increase a proportion of the Corynebacterium microorganisms and Dolosigranulum microorganisms in the nasal microbiota of the individual, relative to a proportion of Moraxella, Staphylococcus, and/or Streptococcus microorganisms in the nasal microbiota of the individual, wherein the individual has asthma or early signs of loss of asthma control.

118. The method of claim 117, wherein the individual is a child.

119. The method of claim 118, wherein the child is receiving a daily inhaled corticosteroid (ICS).

120. The method of claim 117, wherein: the Corynebacterium microorganisms comprise Corynebacterium pseudodiphtheriticum microorganisms and/or Corynebacterium accolens microorganisms; and/or the Dolosigranulum microorganisms comprise Dolosigranulum pigrum microorganisms.

121. The method of claim 120, wherein: the Corynebacterium microorganisms comprise 16S RNA sequences with 99% identity to any one of SEQ ID NO: 7 or 8; and/or the Dolosigranulum microorganisms comprise 16S RNA sequences with 99% identity to SEQ ID NO: 9.

122. The method of claim 117, wherein the bacterial extract is secreted by or obtained from an outer surface of Corynebacterium microorganisms and Dolosigranulum microorganisms.

123. A method comprising:
assaying a biological sample obtained from an individual for a proportion of Corynebacterium microorganisms and Dolosigranulum microorganisms, wherein the individual has asthma or early signs of loss of asthma control; and
administering a therapy to the individual.

124. The method of claim 123, wherein the therapy is a composition comprising Corynebacterium microorganisms and Dolosigranulum microorganisms at a dose sufficient to increase a proportion of the Corynebacterium microorganisms and Dolosigranulum microorganisms in the nasal microbiota of the individual, relative to a proportion of Moraxella, Staphylococcus, and/or Streptococcus microorganisms in the nasal microbiota of the individual.

125. The method of claim 123, wherein the therapy is an inhaled corticosteroid (ICS).

126. The method of claim 123, wherein the biological sample is a nasal sample.

127. The method of claim 123, wherein the individual is a child.

128. The method of claim 123, wherein: the Corynebacterium microorganisms comprise Corynebacterium pseudodiphtheriticum microorganisms and/or Corynebacterium accolens microorganisms; and/or the Dolosigranulum microorganisms comprise Dolosigranulum pigrum microorganisms.

129. The method of claim 128, wherein: the Corynebacterium microorganisms comprise 16S RNA sequences with 99% identity to any one of SEQ ID NO: 7 or 8; and/or the Dolosigranulum microorganisms comprise 16S RNA sequences with 99% identity to SEQ ID NO: 9.

130. The method of claim 124, wherein the composition comprises a bacterial extract secreted by or obtained from an outer surface of the Corynebacterium microorganisms and Dolosigranulum microorganisms.

131. A method comprising:
acquiring a biological sample from an upper airway of an individual with early signs of loss of asthma control;
assaying an amount, concentration, or proportion of Corynebacterium microorganisms and Dolosigranulum microorganisms in the biological sample.

132. The method of claim 131, further comprising assaying an amount, concentration, or proportion of Moraxella, Staphylococcus, and/or Streptococcus microorganisms in the biological sample.

133. The method of claim 132, wherein a greater amount, concentration, or proportion of the Moraxella, Staphylococcus, and/or Streptococcus microorganisms relative to the Corynebacterium and/or Dolosigranulum microorganisms indicates an increased risk of asthma exacerbations in the individual, a decreased time to loss of asthma control or asthma exacerbations in the individual, or an increased risk of loss of asthma control in the individual.

134. The method of claim 131, wherein the biological sample is a nasal blow sample.

135. The method of claim 131, wherein the early signs of loss of asthma control comprise increased use of asthma intervention therapy or increased asthma symptoms.

136. The method of claim 135, wherein the individual with early signs of loss of asthma control used at least one dose of albuterol and remained awake for one night due to asthma symptoms, used at least two doses of albuterol in 6 hours, used at least two inhalations of albuterol in 6 hours, used at least three doses of albuterol in 24 hours, or used at least 6 inhalations of albuterol in 24 hours.


SEQUENCE LISTING (11 sequences)

SEQ ID NO: 1 - DNA, 480 bp, Moraxella catarrhalis
attgaacgctggcggcaggcttaacacatgcaagtcgaacgaagttaggaagcttgcttctgatacttagtggcggacgggtgagtaatgcttaggaatctgcctagtagtgggggataacttggggaaacccaagctaataccgcatacgacctacgggtgaaagggggcttttagctctcgctattagatgagcctaagtcggattagctggttggtggggtaaaggcctaccaaggcgacgatctgtagctggtctgagaggatgatcagccacactgggactgagacacggcccagactcctacgggaggcagcagtggggaatattggacaatgggcgaaagcctgatccagccatgccgcgtgtgtgaagaaggccttttggttgtaaagcactttaagtggggaggaaaagcttatggttaatacccataagccctgacgttacccacagaataagcaccggctaactctgtg

SEQ ID NO: 2 - DNA, 479 bp, Moraxella nonliquefaciens
attgaacgctggcggcaggcttaacacatgcaagtcgaacgatgaagtctagcttgctagacggattagtggcgaacgggtgagtaatgcttaggaatctgcctattagtgggggataacgtagggaaacttacgctaataccgcatacgacctacgggtgaaagggggcgtttagctctcgctaatagatgagcctaagtcggattagctagttggtggggtaaaggcctaccaaggcgacgatctgtagctggtctgagaggatgatcagccacactgggactgagacacggcccagactcctacgggaggcagcagtggggaatattggacaatgggcgaaagcctgatccagccatgccgcgtgtgtgaagaaggccttttggttgtaaagcactttaagtggggaggaaaagcttgtggttaatacccataagccctgacgttacccacagaataagcaccggctaactctgtg

SEQ ID NO: 3 - DNA, 478 bp, Moraxella lincolnii
attgaacgctggcggcaggcttaacacatgcaagtcgaacgaagaggtctagcttgctagactgattagtggcgaacgggtgagtaacatttaggaatctaccttatagagggggatagctcggggaaactcgaattaataccgcatacgacctacgggtgaaagggggcgcaagctcttgctataagatgagcctaaaccagattagctagttggtggggtaaaggcctaccaaggcgacgatctgtagctggtctgagaggatgatcagccacactgggactgagacacggcccagactcctacgggaggcagcagtggggaatattggacaatgggcgaaagcctgatccagccatgccgcgtgtgtgaagaaggccttatggttgtaaagcactttaagtggggaggaaaagcttatggataacacccataggttctgacgttacccacagaataagcaccggctaactctgtg

SEQ ID NO: 4 - DNA, 497 bp, Staphylococcus aureus
gatgaacgctggcggcgtgcctaatacatgcaagtcgagcgaacggacgagaagcttgcttctctgatgttagcggcggacgggtgagtaacacgtggataacctacctataagactgggataacttcgggaaaccggagctaataccggataatattttgaaccgcatggttcaaaagtgaaagacggtcttgctgtcacttatagatggatccgcgctgcattagctagttggtaaggtaacggcttaccaaggcaacgatgcatagccgacctgagagggtgatcggccacactggaactgagacacggtccagactcctacgggaggcagcagtagggaatcttccgcaatgggcgaaagcctgacggagcaacgccgcgtgagtgatgaaggtcttcggatcgtaaaactctgttattagggaagaacatatgtgtaagtaactgtgcacatcttgacggtacctaatcagaaagccacggctaactacgtg

SEQ ID NO: 5 - DNA, 497 bp, Staphylococcus epidermidis
gatgaacgctggcggcgtgcctaatacatgcaagtcgagcgaacagatgaggagcttgctcctctgacgttagcggcggacgggtgagtaacacgtggataacctacctataagactgggataacttcgggaaaccggagctaataccggataatatattgaaccgcatggttcaatagtgaaagacggttttgctgtcacttatagatggatccgcgccgcattagctagttggtaaggtaacggcttaccaaggcgacgatgcgtagccgacctgagagggtgatcggccacactggaactgagacacggtccagactcctacgggaggcagcagtagggaatcttccgcaatgggcgaaagcctgacggagcaacgccgcgtgagtgatgaaggtcttcggattgtaaaactctgttattagggaagaacaaatgtgtaagtaactatgcacgtcttgacggtacctaatcagaaagccacggctaactacgtg

SEQ ID NO: 6 - DNA, 495 bp, Streptococcus pneumoniae
gacgaacgctggcggcgtgcctaatacatgcaagtagaacgctgaaggaggagcttgcttctctggatgagttgcgaacgggtgagtaacgcgtaggtaacctgcctggtagcgggggataactattggaaacgatagctaataccgcataagagtagatgttgcatgacatttgcttaaaaggtgcacttgcatcactaccagatggacctgcgttgtattagctagttggtggggtaacggctcaccaaggcgacgatacatagccgacctgagagggtgatcggccacactgggactgagacacggcccagactcctacgggaggcagcagtagggaatcttcggcaatggacggaagtctgaccgagcaacgccgcgtgagtgaagaaggttttcggatcgtaaagctctgttgtaagagaagaacgagtgtgagagtggaaagttcacactgtgacggtatcttaccagaaagggacggctaactacgtg

SEQ ID NO: 7 - DNA, 463 bp, Corynebacterium pseudodiphtheriticum
gacgaacgctggcggcgtgcttaacacatgcaagtcgaacggaaaggcctcttcggaggtactcgagtggcgaacgggtgagtaacacgtgggtgatctgccctgcactctgggataagcctgggaaactgggtctaataccggataggaccatgctttagtgtgtgtggtggaaagttttttcggtgtaggatgagcccgcggcctatcagcttgttggtggggtaatggcctaccaaggcggcgacgggtagccggactgagaggttggtcggccacattgggactgagatacggcccagactcctacgggaggcagcagtggggaatattgcacaatgggcgcaagcctgatgcagcgacgccgcgtgggggatgacggccttcgggttgtaaactcctttcgccagggacgaagcgttttgtgacggtacctggagaagaagcaccggctaactacgtg

SEQ ID NO: 8 - DNA, 465 bp, Corynebacterium accolens
gacgaacgctggcggcgtgcttaacacatgcaagtcgaacggaaaggccctgcttgcagggtactcgagtggcgaacgggtgagtaacacgtgggtgatctgccctgcacttcgggataagcttgggaaactgggtctaataccggataggagccatctttagtgtggtggttggaaagttttttcggtgtaggatgagctcgcggcctatcagcttgttggtggggtaatggcctaccaaggcggcgacgggtagccggcctgagagggtgtacggccacattgggactgagatacggcccagactcctacgggaggcagcagtggggaatattgcacaatgggcgcaagcctgatgcagcgacgccgcgtgggggatgaaggccttcgggttgtaaactcctttcgctagggacgaagctttttgtgacggtacctagataagaagcaccggctaactacgtg

SEQ ID NO: 9 - DNA, 494 bp, Dolosigranulum pigrum
gacgaacgctggcggcatgcctaatacatgcaagtcgaacgatgatatcactgcttgcagtgattgattagtggcgaacgggtgagtaacacgtgaggaacttgcccatgagcgggggacaacattcggaaacggatgctaataccccataggtggattggtcgcatgacgaattcattaaaggtggctttgctaccactcatggatagcctcgcggcgtattagctagttggtaaggtaatggcttaccaaggcagtgatacgtagccgacttgagagggtgatcggccacactgggactgagacacggcccagactcctacgggaggcagcagtagggaatcttccacaatgggtgcaaacctgatggagcaatgccgcgtgagtgaagaaggtcttcggatcgtaaagctctgttgttagagaagaacacgtgctaggtaactactagcgccttgacggtatctaaccagaaagtcacggctaactacgtg

SEQ ID NO: 10 - DNA, 20 bp, Artificial Sequence
  Synthetic
agagtttgatcctggctcag

SEQ ID NO: 11 - DNA, 17 bp, Artificial Sequence
  Synthetic
attaccgcggctgctgg